SEQUENCE LISTING (41 sequences)

SEQ ID NO: 1 - DNA, 48 bp, Artificial
  Synthetic Primer
ggtccgactaccccaacaataccttgaaccctacagcagagtctgcat

SEQ ID NO: 2 - DNA, 18 bp, Artificial
  Synthetic Primer
gcgccctggtggaagaca

SEQ ID NO: 3 - DNA, 15 bp, Artificial
  Synthetic Primer
gcttccaggcccgcc

SEQ ID NO: 4 - DNA, 18 bp, Artificial
  Synthetic Primer
gccaagctggaggagcag

SEQ ID NO: 5 - DNA, 17 bp, Artificial
  Synthetic Primer
ggcagtgtaccaggccg

SEQ ID NO: 6 - DNA, 17 bp, Artificial
  Synthetic Primer
ggagccgactggccaat

SEQ ID NO: 7 - DNA, 19 bp, Artificial
  Synthetic Primer
ggtccgactaccccaacaa

SEQ ID NO: 8 - DNA, 20 bp, Artificial
  Synthetic Primer
cttgaaccctacagcagagt

SEQ ID NO: 9 - DNA, 23 bp, Artificial
  Synthetic Primer
caccgttagtacgagtgtcggtg

SEQ ID NO: 10 - DNA, 53 bp, Artificial
  Synthetic Primer
ggtccgactaccccaacaataccaccgttagtacgagtgtcggtgtgagctac

SEQ ID NO: 11 - DNA, 19 bp, Artificial
  Synthetic Primer
cgcgcctgagatgaagcac

SEQ ID NO: 12 - DNA, 54 bp, Artificial
  Synthetic Primer
ggtccgactaccccaacaataccaccgttagtacgagtgtcggtgcagactccg

SEQ ID NO: 13 - DNA, 18 bp, Artificial
  Synthetic Primer
cgcgcctccttcattcca

SEQ ID NO: 14 - DNA, 48 bp, Artificial
  Synthetic Primer
ggtccgactaccccaacaataccttgaaccctacagcagagtcacatg

SEQ ID NO: 15 - DNA, 52 bp, Artificial
  Synthetic Primer
ggtccgactaccccaacaataccttgaaccctacagcagagtttccacatgc

SEQ ID NO: 16 - DNA, 19 bp, Artificial
  Synthetic Primer
cctgatgccagtgggcagt

SEQ ID NO: 17 - DNA, 18 bp, Artificial
  Synthetic Primer
tccctggccatccatgtg

SEQ ID NO: 18 - DNA, 24 bp, Artificial
  Synthetic Primer
ggaaagaggcagtcttcttagcat

SEQ ID NO: 19 - DNA, 24 bp, Artificial
  Synthetic Primer
cgagctagttaaacagtgccattg

SEQ ID NO: 20 - DNA, 22 bp, Artificial
  Synthetic Primer
ggaaagcctcatttgtgtggaa

SEQ ID NO: 21 - DNA, 19 bp, Artificial
  Synthetic Primer
ggtccgactaccccaacaa

SEQ ID NO: 22 - DNA, 19 bp, Artificial
  Synthetic Primer
cgcgcctgagatgaagcac

SEQ ID NO: 23 - DNA, 18 bp, Artificial
  Synthetic Primer
gcgcctgagatgaagcac

SEQ ID NO: 24 - DNA, 17 bp, Artificial
  Synthetic Primer
cgcctgagatgaagcac

SEQ ID NO: 25 - DNA, 16 bp, Artificial
  Synthetic Primer
gcctgagatgaagcac

SEQ ID NO: 26 - DNA, 15 bp, Artificial
  Synthetic Primer
cctgagatgaagcac

SEQ ID NO: 27 - DNA, 14 bp, Artificial
  Synthetic Primer
ctgagatgaagcac

SEQ ID NO: 28 - DNA, 14 bp, Artificial
  Synthetic Primer
gtgagatgaagcac

SEQ ID NO: 29 - DNA, 13 bp, Artificial
  Synthetic Primer
tgagatgaagcac

SEQ ID NO: 30 - DNA, 18 bp, Artificial
  Synthetic Primer
cgcgcctccttcattcca

SEQ ID NO: 31 - DNA, 17 bp, Artificial
  Synthetic Primer
gcgcctccttcattcca

SEQ ID NO: 32 - DNA, 16 bp, Artificial
  Synthetic Primer
cgcctccttcattcca

SEQ ID NO: 33 - DNA, 15 bp, Artificial
  Synthetic Primer
gcctccttcattcca

SEQ ID NO: 34 - DNA, 14 bp, Artificial
  Synthetic Primer
cctccttcattcca

SEQ ID NO: 35 - DNA, 13 bp, Artificial
  Synthetic Primer
ctccttcattcca

SEQ ID NO: 36 - DNA, 12 bp, Artificial
  Synthetic Primer
tccttcattcca

SEQ ID NO: 37 - DNA, 15 bp, Artificial
  Synthetic Primer
tatgagatgaagcac

SEQ ID NO: 38 - DNA, 17 bp, Artificial
  Synthetic Primer
tatatgagatgaagcac

SEQ ID NO: 39 - DNA, 26 bp, Artificial
  Synthetic Primer
atatatatatatatgagatgaagcac

SEQ ID NO: 40 - DNA, 44 bp, Artificial
  Synthetic Primer
atatatatatatatatatatatatatatatatgagatgaagcac

SEQ ID NO: 41 - DNA, 60 bp, Artificial
  Synthetic Primer
atatatatatatatatatatatatatatatatatatatatatatatatgagatgaagcac

(19) United States
(12) Patent Application Publication
(10) Pub. No.: US 2008/0131878 A1
(43) Pub. Date: Jun. 5, 2008

(54) Compositions and Methods for the Detection of Small RNA

(75) Inventors: Gary J. Latham, Austin, TX (US); Jon Kemppainen, Austin, TX (US)

Correspondence Address: Fullbright & Jaworski L.L.P., 600 Congress Avenue, Suite 2400, Austin, TX, 78701, US

(73) Assignee: Asuragen, Inc.

(21) Appl. No.: 11/567,082

(22) Filed: Dec. 5, 2006

(51) Int. Cl. C12Q 1/68; C07H 21/02

(52) U.S. Cl. 435/6; 536/23.1; 536/24.31; 536/24.33

(57) ABSTRACT

Embodiments of the invention include methods of detecting one or more RNA by reverse transcribing one or more RNA target using one or more reverse transcription primer comprising in a 5′ to 3′ direction (i) a primer segment, (ii) a probe segment distinct from the primer segment, and (iii) a 3′ target specific segment that anneals to a RNA target; amplifying one or more RNA from the reverse transcription reaction using a first amplification primer that anneals to the 3′ end of a reverse transcribed RNA target and a second primer that anneals to a sequence complementary to the primer segment; and detecting amplification of a target nucleic acid.

The government owns rights in the invention pursuant to a Phase II SBIR grant number 2R44GM072391.

### BACKGROUND OF THE INVENTION

I. Field of the Invention

The present invention relates to the fields of molecular biology. In particular, the invention relates to compositions and methods for the detection of RNA and small RNA.

II. Background

In 2001, several groups used a cloning method to isolate and identify a large group of small RNAs, “microRNAs” (miRNAs), from C. elegans, Drosophila, and humans (Lagos-Quintana et al., 2001; Lau et al., 2001; Lee and Ambros, 2001). Several hundreds of miRNAs have been identified in plants and animals, including humans. During the past five years, miRNAs have emerged as a critical new class of mammalian cell regulatory molecules and have been implicated in diverse cellular and biological processes such as apoptosis, proliferation, epithelial cell morphogenesis, neural and muscle cell differentiation, fat/cholesterol/glucose homeostasis, and viral infection. As global regulators of diverse biological processes, miRNAs may be important diagnostic analytes.

Because miRNAs are extremely short, existing strategies that were optimized for the isolation and detection of longer mRNA species have not proved applicable to miRNA analysis. In some instances, a major disadvantage of existing approaches is that insufficient sequence space is provided in small RNA targets. For example, the method of choice for the sensitive and specific detection of mRNA has been quantitative RT-PCR (qRT-PCR) using dual-labeled probes. In this case, two primers and a non-overlapping probe sequence are used to ensure highly specific target detection and accurate quantification. Yet, the accepted PCR cycling parameters require that amplicons of at least 50-60 nucleotides be used to accommodate this conventional design. Thus, miRNAs are too short to be amenable to the standard qRT-PCR approach.

Several strategies have been described for the detection of miRNAs including conventional northern blot analysis, ribonuclease protection assays (Ambion), microarrays (Ambion, Invitrogen, Genisphere), bead-based hybridization schemes (Luminex), and qRT-PCR (ABI). The direct detection methodologies suffer from relatively low sensitivity. Indirect detection methodologies are more sensitive, but in their current format, many variations are not suited for incorporation into a diagnostic assay. In particular, high background problems can significantly impair the ability of these assays to quantify certain miRNA targets. qRT-PCR formats that detect amplification products using non-specific DNA binding dyes are especially susceptible to these background problem.

One strategy for detection and quantification of miRNAs uses qRT-PCR in conjunction with a TaqMan probe partially complementary to a specific miRNA target and partially complementary to a hairpin RT primer (U.S. Publication 20050266418). A limitation of this method is that, like essentially all PCR strategies using target sequence-specific reporters, a unique probe sequence must be designed and synthesized for each miRNA target of interest. This process is time-consuming and costly. Also, because probe sequences are target-defined, the context of some sequences will be less amenable than others for enabling optimal assay performance.

To address some of the problems associated with target-specific probes, universal reporter probes have been employed. One such qRT-PCR method has been described for the detection of DNA mutations. This method (Whitcombe et al., 1998) utilizes a universal probe that is contained within the “flap” region (i.e., that region which is 5′ of the gene-hybridizing region) of a PCR forward primer for the purpose of detecting DNA mutations via ARMS (amplification refractory mutation system). In this case, an additional, universal forward primer was employed to prevent direct hybridization of the probe to the primer. The universal primer was designed to have a higher Tm than the gene-specific primer, thus encouraging the more selective use of the universal primer following an increase in the cycling temperature. Extension by the universal forward primer was also favored by the use of at least a 20-fold higher concentration compared to the gene-specific primer (0.5 μM vs. 10-25 nM). A similar strategy (Rickert et al., 2004) employed a low concentration of the gene-specific forward primer containing a complementary sequence to the universal TaqMan™ probe, and a much higher concentration of the universal forward primer, which would then control the PCR after the gene-specific forward sequence had been immortalized in the target amplicon. This strategy was used to measure the differential expression of FLJ10350, TNNI1, and PIPPIN in biosamples from patients suffering from congenital heart defects, whereby the universal probe enabled detection in the PCR step following reverse transcription of the RNA.

Thus, there remains a need for detection of RNA in samples and in particular small RNA, such as miRNA.

### SUMMARY OF THE INVENTION

The present invention employs reverse transcription coupled with quantitative PCR, in which a non-target probe sequence (i.e., sequence not present in the target RNA) is defined within the reverse transcription primer. Certain embodiments of the invention describe a method for detection and/or quantification of small RNAs, such as miRNAs, that requires as few as three oligonucleotide primers (one for reverse transcription and two for quantitative PCR). The invention describes assays and assay methods that may use fewer components, and may, but need not provide for simpler optimization and lower overall costs. Certain non-limiting aspects of the invention include methods that reduce or eliminate target-independent signal generation. Embodiments of the invention may use fewer reaction components, producing a lower background signal, and may exhibit improved sensitivity and specificity, as well as provide for simpler optimization. Other embodiments of the invention are suitable for use in diagnostic and prognostic assays, particularly in clinical samples. Such samples may contain degraded or modified RNA for which detecting amplicons of limited size would be beneficial. Embodiments of the invention can detect nucleic acids of 18 nucleotides or less.

Embodiments of the invention include methods of detecting one or more RNA comprising the steps of: (a) reverse transcribing one or more RNA target using one or more reverse transcription primer comprising in a 5′ to 3′ direction (i) a primer segment, (ii) a probe segment, and (iii) a 3′ target specific segment that anneals to a RNA target; (b) amplifying one or more RNA or RNA segment from all or part of the reverse transcription reaction using a first amplification primer that anneals to the 3′ end of a reverse transcribed RNA target and a second primer that anneals to a sequence complementary to the primer segment; and (c) detecting amplification of a target nucleic acid. Typically, but not necessarily, one or more segment of the RT primer is distinct (i.e., not overlapping in sequence) from other segments of the RT primer. RNA targets include, but are not limited to, small RNAs, such as miRNA, siRNA; piwi interacting RNA (Girard et al., 2006); mRNA; rRNA, and the like. The RNA target may be present in less than 1,000,000, 100,000, 10,000, 5,000, 2,500, 1,000, 500, 100 copies or copies per cell.

In certain aspects, the 3′ target specific segment of the reverse transcription primer anneals to a contiguous sequence of about, at least about or at most about 4, 5, 6, 7, 8, 9, 10 to 11, 12, 13, 14, 15, 16, 17, 18, 19, 20 or more nucleotides of the RNA target.

In further aspects, the first amplification primer is present at a concentration of about, at least about, or at most about 10, 50, 100, 150, 200, 250, 300, 350 to 300, 350, 400, 450, 500, 550, 600, 800, 10000 nM or μM, or any range or value derivable there between, and the second amplification primer is present at a concentration of 50, 100, 150, 200, 250, 300, 350 to 300, 350, 400, 450, 500, 550, 600, 650, 700, 750, 800, 1000 nM or μM, or any range or value derivable there between. Detecting amplification can comprise detecting association of a probe with a sequence of the probe segment or a complement to the probe segment. A probe may be a 5′-exonuclease assay probe, stem-loop molecular beacon, stemless or linear beacon, PNA Molecular Beacon, linear PNA beacon, non-FRET probe, Sunrise®/Amplifluor® (probe, stem-loop and duplex Scorpion™ probe, bulge loop probe, pseudo knot probe, cyclicon, MGB Eclipse™ probe, hairpin probe, peptide nucleic acid (PNA) light-up probe, self-assembled nanoparticle probe, or ferrocene-modified probe. In certain embodiments the probe is a 5′exonuclease probe or a beacon probe.

In another aspect, the RNA can be at least about or at most about 10, 20, 30, 40, 50, 60, 70, 80, 90, 100, 200, 300, 400, 500 base pairs, or 1, 2, 3, 4, 5 kilobases or more in length including all values and ranges there between. In certain aspects, the first amplification primer comprises a 5′ non-complementary sequence of 1, 2, 3, 4, 5, 6, 7, 8, 9 to 10, 15, 20, 25, 30, or more nucleotides, including all values and ranges there between. In certain embodiments, the first amplification primer comprises a 5′ non-complimentary sequence of 2-4 to 8-10 nucleotides. The non-complimentary sequence can comprise a number of different sequences in particular, the non-complementary sequence may comprise 50, 60, 70, 80, 90, or 100% cytosine and/or guanine residues.

Embodiments of the invention may further comprise diluting the reverse transcription reaction prior to amplification 0.5, 1, 2, 5, 10, 50, 100, 200, 400, 800, 1000, 5000 or more fold, including all values and ranges there between. In certain aspects, the reverse transcription reaction is diluted 2 to 100 fold.

Further embodiments of the invention include methods of detecting a miRNA in a sample comprising the steps of (a) obtaining a RNA sample; (b) reverse transcribing one or more miRNA target in the RNA sample using one or more reverse transcription primer comprising in a 5′ to 3′ direction (i) a primer segment, (ii) a probe segment, which may or may not be distinct from the primer segment, and (iii) a 3′ target specific segment that anneals to a RNA target; (c) amplifying the product of the reverse transcription reaction using a first primer that anneals to the 3′ portion of a reverse transcribed target miRNA and a second primer that anneals to a sequence complementary to the primer segment; and (d) detecting amplification of the probe segment. In certain aspects, the sample is a biopsy sample, a histological sample, or a fluid sample.

In still further embodiments, the invention includes a method of diagnosing a pathological condition comprising the steps of (a) reverse transcribing RNA in a RNA sample from a subject having, suspected of having, or at risk of developing a pathological condition using a reverse transcription primer specific for one or more RNA associated with one or more pathological condition; (b) amplifying the product of the reverse transcription reaction using a first primer that anneals to the 5′ portion of a target RNA and a second primer that anneals to the universal primer segment of the reverse transcription primer; and (c) detecting amplification of the probe segment of the reverse transcription primer. The RNA can be any RNA, including, but not limited to a small RNA, a miRNA, rRNA, tRNA, mRNA, siRNA and the like. A sample can be a biopsy sample, a histological sample, or a biological fluid.

Embodiments of the invention also include nucleic acid amplification kits comprising a reverse transcription primer comprising in the 5′ to 3′ direction (a) a primer segment; (b) a probe segment; and (c) a target segment of 5, 6, 7, 8 to 9, 10, 11, 12 or more nucleotides that are complementary to a nucleic acid sequence in a RNA target. The kit may further comprise a first amplification primer that anneals to a complementary sequence in a target RNA and a second primer that anneals to a complementary sequence in a primer segment present in a reverse transcription primer.

Further embodiments of the invention include a reverse transcription primer comprising in a 5′ to 3′ direction (a) a primer segment; (b) a probe segment the complement of which is detectable by a sequence specific probe; and (c) a target segment of 5, 6, 7, 8 to 9, 10, 11, 12 or more nucleotides that is complementary to a RNA target.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

As used herein, the terms “complementary” or “complementarity” are used in reference to polynucleotides (i.e., a sequence of nucleotides) related by the base-pairing rules. For example, for the sequence “5′-A-G-T-3′” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, “reagents” for any enzymatic reaction mixture, such as a reverse transcription and PCR reaction mixture, are any compound or composition that is added to the reaction mixture including, without limitation, enzyme(s), nucleotides or analogs thereof, primers and primer sets, buffers, salts and co-factors. As used herein, unless expressed otherwise, “reaction mixture” includes all necessary compounds and/or compositions necessary to perform that enzymatic reaction, even if those compounds or compositions are not expressly indicated.

A “probe” is a polynucleotide that is capable of binding to a complementary target nucleic acid sequence. In certain embodiments, the probe is used to detect amplified target nucleic acid sequences. In certain embodiments, the probe incorporates a label.

The term “label” refers to any molecule that can be detected. In certain embodiments, a label can be a moiety that produces a signal or that interacts with another moiety to produce a signal. In certain embodiments, a label can interact with another moiety to modify a signal of the other moiety. In certain embodiments, a label can bind to another moiety or complex that produces a signal or that interacts with another moiety to produce a signal. In certain embodiments, the label emits a detectable signal only when the probe is bound to a complementary target nucleic acid sequence. In certain embodiments, the label emits a detectable signal only when the label is cleaved from the polynucleotide probe. In certain embodiments, the label emits a detectable signal only when the label is cleaved from the polynucleotide probe by a 5′ exonuclease reaction.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic representation of various aspects of certain embodiments of the invention. For detecting a small RNA target, a reverse transcription (RT) primer containing a target annealing region, a universal probe sequence upstream of the annealing region, and a universal reverse-PCR primer sequence upstream of the universal probe sequence, is annealed to the small RNA target. Following reverse transcription, PCR is performed for amplification, detection, and quantification of the small RNA target. PCR utilizes a small RNA-specific forward primer with an annealing region and an optional non-complementary or “flap” sequence upstream of the annealing region. PCR also utilizes a universal reverse PCR primer whose sequence is defined by the RT primer and a universal TaqMan™ probe, whose sequence is also defined by the RT primer. The uncleaved TaqMan™ probe contains a quenching moiety (Q) and a fluorescent moiety (F).

FIG. 2. Specificity of amplification with RT primers having six or ten nucleotides complementary to the target RNA. Experimental methods are described in Example 4. RT reactions contained no RNA (NTC), 500 ng RNA, or 5 ng RNA. Amplicon size: 1, 26 nt; 2, 65 nt; 3, 129 nt; 4, 331 nt; 5, 789 nt. Top panel; reaction products following reverse transcription with RT primer having 6 nt complementary to target RNA. Bottom panel; reaction products following reverse transcription with RT primer having 10 nt complementary to target RNA.

FIG. 3. Improved specificity of amplification with an RT primer having more than eight nucleotides complementary to the target RNA. Experimental methods are described in Example 5.

FIG. 4. Sensitive and specific detection of five miRNAs in various input amounts of human pancreas total RNA. Experimental methods are described in Example 15.

FIG. 5. Quantification of mature miRNA in the presence of precursor miRNA. Experimental methods are described in Example 16.

FIG. 6. Quantification of miRNA using a universal probe defined in the RT primer is superior to quantification using a universal probe defined in the forward (FW) PCR primer. Experimental methods are described in Example 19.

### DETAILED DESCRIPTION OF THE INVENTION

A typical PCR reaction includes multiple amplification steps, or cycles that selectively amplify a target nucleic acid species. A general description of the PCR process, and common variations thereof, such as quantitative PCR (qPCR), real-time qPCR, reverse transcription PCR (RT-PCR) and quantitative reverse transcription PCR (qRT-PCR) are well-described in the art and have been broadly commercialized.

### I. Methods of Detecting RNA

Reverse transcription (RT) and the polymerase chain reaction (PCR) are critical to many molecular biology and related applications, particularly gene expression analysis. In these applications, reverse transcription is used to prepare template DNA from an initial RNA sample. The template DNA can then be amplified using PCR to produce a sufficient amount of amplified product for the application of interest. Advances in nucleic acid extraction and amplification have greatly expanded the types of biological samples from which genetic material may be obtained. In particular, PCR has made it possible to obtain sufficient quantities of DNA from fixed tissue samples, archaeological specimens, and quantities of many types of cells that number in the single digits. Detecting, analyzing, and/or quantifying small RNA requires the amplification and detection of RNA with a limited size posing difficulty in analysis of these important RNA targets.

As described herein in more detail, aspects of the methods include detecting one or more RNA comprising the steps of: (a) reverse transcribing one or more RNA target using one or more reverse transcription primer comprising in a 5′ to 3′ direction (i) a primer segment, (ii) a probe segment, and (iii) a 3′ target specific segment that anneals to a RNA target; (b) amplifying one or more RNA or RNA segment from all or part of the reverse transcription reaction using a first amplification primer that anneals to the 3′ end of a reverse transcribed RNA target and a second primer that anneals to a sequence complementary to the primer segment; and (c) detecting amplification of a target nucleic acid.

A. Reverse Transcription Reaction

Typically, the components of a reverse transcription reaction, e.g., nuclease free water, RT buffer, dNTP mix, RT primer, RNase inhibitor, and a reverse transcriptase, are assembled on ice prior to the addition of a RNA template. An example of a RT reaction may include a 1× final concentration of RT buffer, a 1 mM final concentration of dNTPs, a 50 nM final concentration of RT primer, an effective amount of a RNase inhibitor(s), an amount of a reverse transcriptase or equivalent enzyme sufficient to produce a DNA template, a particular mass of template RNA in an appropriate volume and nuclease free water to bring the reaction to a particular volume, such as 5, 10, 15, 20, 25, 50, 100 μl total volume or any volume or range of volumes there between. Following assembly of the reaction components, at least about, at most about or about 1, 5, 10, 20, 30, 40, 50, 100, 200 or more pg or ng of a RNA template are added to the reaction mix. If a RNA template is a synthetic RNA, a background of 10 ng/μl of non-target RNA or nucleic acid, such as polyA RNA, can be added. The reverse transcription reaction is typically incubated at least, at most, or at about 5, 10, 11, 12, 13, 14, 15, 16, 17, 18, 19, 20, 25, 30, 32, or 35° C. for 5, 10, 15, 20, 25, 60, 120 minutes or more, then at 20, 25, 30, 32, 35, 40, 41, 42, 43, 44, 45, or 50° C., including all temperatures there between, for 10, 15, 20, 25, 30, 35, 40, 45, 50, 60 min, including all times there between, then at 70, 75, 80, 85, 90, 95, 100, 110° C., including all temperatures there between for 2, 3, 4, 5 to 10, 20, 30 minutes, including all times there between.

1. Reverse Transcription Primer

The reverse transcription primer typically comprises in a 5′ to 3′ direction (i) a primer segment, (ii) a probe segment, and (iii) a 3′ target specific segment that anneals to an RNA target. The primer can be a unique primer segment. In certain embodiments the primer segment can be a universal primer segment, that is a segment that corresponds to a primer that can be used to prime 2, 3, 4, 5, 6, or more different amplicons, or RNA targets or segments of RNA targets, that is a primer that is not specific for a target RNA. The primer segment can be from 5, 6,7, 8,9, 10, 11, 12, 13, 14, 15, 16, 17, 18, 19, 20, 21, 22,23,24,25, 26,27,28,29,30,40,50, 100 or more nucleotides in length, including all values and ranges there between.

The probe segment will typically be distinct from a primer segment and/or the target specific segment of the RT primer. The probe segment can be from 5, 6, 7, 8, 9 10, 11, 12, 13, 14, 15, 16, 17, 18, 19, 20. 21. 22. 23. 24. 25. 26, 27, 28, 29, 30, 40, 50, 100 or more nucleotides in length, including all values and ranges there between. In certain aspects the probe will be adjacent to the primer segment, the target specific segment or both the primer segment and the target specific segment.

The 3′ target specific segment comprises a sequence that anneals to a target RNA sequence or its complement and can be from 5, 6, 7, 8, 9, 10, 11, 12, 13, 14, 15, 16, 17, 18, 19, 20, 21, 22, 23, 24, 25, 26, 27, 28, 29, 30, 40, 50, 100 or more nucleotides in length, including all values and ranges there between. The target segment may contain modified bases, such as locked nucleic acid (LNA), 2-O-alkyl, 5′ propyne, G-clamp, or other modified bases. Such bases are typically used to improve binding affinity to the target.

2. Target Nucleic Acids

In certain embodiments, target nucleic acid sequences include RNA that includes, but are not limited to, mRNA, miRNA, siRNA, piwi-interacting RNA, rRNA, tRNA, snRNA, viral RNA and fragments and segments thereof.

A variety of methods are available for obtaining a target nucleic acid sequence. When the nucleic acid target is obtained through isolation from a biological matrix, certain isolation techniques include, but are not limited to, (1) organic extraction followed by ethanol precipitation, e.g., using a phenol/chloroform organic reagent (Ausubel et al., 1993), in certain embodiments, using an automated nucleic acid extractor, e.g., the Model 341 DNA Extractor available from Applied Biosystems (Foster City, Calif.); (2) stationary phase adsorption methods (U.S. Pat. No. 5,234,809; Walsh et al., 1991); and (3) salt-induced nucleic acid precipitation methods (Miller et al., (1988), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. Pat. No. 7,001,724.

In certain embodiments, a target nucleic acid sequence may be derived from any living, or once living, organism, including but not limited to, a prokaryote, a eukaryote, a plant, an animal, a human, and a virus. In certain embodiments, a target nucleic acid sequence is derived from a human. In certain embodiments, a RNA may be reverse-transcribed into a DNA target nucleic acid sequence.

In certain embodiments, multiple target nucleic acid sequences can be amplified in the same reaction (e.g., in multiplex amplification reactions). Aspects of the invention may include 1, 2, 3, 4, 5, 6, 7, 8, 9, 10 or more different amplifications in a reaction.

Different target nucleic acid sequences may be different portions of a single contiguous nucleic acid or may be on different nucleic acids. Different portions of a single contiguous nucleic acid may or may not overlap.

In certain aspects, a target nucleic acid sequence is derived from a crude cell lysate. Examples of target nucleic acid sequences include, but are not limited to, nucleic acids from buccal swabs, crude bacterial lysates, blood, skin, semen, hair, bone, mucus, saliva, cell cultures, and tissue biopsies. In still further aspects, target nucleic acid sequences are obtained from a cell, cell line, tissue, or organism that has undergone a treatment, is suspected of contributing or having the propensity of contributing to a pathological condition or is diagnostic of a pathological condition or the risk of developing a pathological condition. In certain embodiments, the methods detect the presence, absence, up-regulation, or down-regulation of certain target nucleic acid sequences in treated cells, cell lines, tissues, or organisms.

In yet further aspects, a target nucleic acid sequence(s) is obtained from a single cell, tens of cells, hundreds of cells or more. In some aspects, a target nucleic acid sequence is extracted from cells of a single organism. In other aspects, a target nucleic acid sequence is extracted from cells of two or more different organisms. A target nucleic acid sequence concentration in a PCR reaction may range from about 1, 100, 1,000 to about 100,000, 1,000,000, 10,000,000 molecules per reaction, including all values there between.

Certain embodiments of the invention are directed to detection and quantitation of miRNA. MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) become part of a large complex to down-regulate a particular target gene. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

miRNAs can be employed in diagnostic, therapeutic, or prognostic applications, particularly those related to pathological conditions described herein. They may be isolated and/or purified. The term “miRNA,” includes the processed RNA and its precursor.

Target RNA may be at least, at most, or about 3, 4, 5, 6, 7, 8, 9, 10, 11, 12, 13, 14, 15, 16, 17, 18, 19, 20, 21, 22,23,24,25,26,27,28,29,30,31,32,33,34,35,36,37,38,39,40,41, 42,43,44,45,46,47,48,49,50,51,52,53,54,55,56,57,58,59,60,61,62,63,64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97,98,99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides or kilobases, or any range derivable therein, in length. In many embodiments, miRNA are 19-24 nucleotides in length depending on the length of the processed miRNA and any flanking regions added. miRNA precursors are generally between 62 and 110 nucleotides in humans.

It is understood that a miRNA is derived from genomic sequences or a gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

3. Reverse Transcriptase (RT)

As used herein, the term “reverse transcriptase (RT)” is used in its broadest sense to refer to any enzyme that exhibits reverse transcription activity as measured by methods known in the art. Reverse transcriptase activity refers to the ability of an enzyme to synthesize a DNA strand utilizing an RNA strand as a template. A “reverse transcriptase” of the present invention, therefore, includes reverse transcriptases from retroviruses, other viruses, and bacteria, as well as a DNA polymerase exhibiting reverse transcriptase activity, such as Tth DNA polymerase, Taq DNA polymerase, Tne DNA polymerase, Tma DNA polymerase, etc. RT from retroviruses include, but are not limited to, Moloney Murine Leukemia Virus (M-MLV) RT, Human Immunodeficiency Virus (HIV) RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT, and as described in U.S. patent application 2003/0198944 (hereby incorporated by reference in its entirety). For review, see e.g. Levin (1997); Brosius et al. (1995). Reverse transcriptase has been used primarily to transcribe RNA into cDNA, which can then be cloned into a vector for further manipulation or used in various amplification methods such as polymerase chain reaction (PCR), nucleic acid sequence-based amplification (NASBA), transcription mediated amplification (TMA), or self-sustained sequence replication (3SR). Typically, any endogenous RNaseH activity has been modified or removed from an enzyme used in the reverse transcription reaction.

B. Amplification Reactions

A typical polymerase chain reaction (PCR) includes three steps: a denaturing step in which a target nucleic acid is denatured; an annealing step in which a set of PCR primers (forward and reverse (backward) primers) anneal to complementary DNA strands; and an elongation step in which a thermostable DNA polymerase elongates the primers. By repeating this step multiple times, a DNA fragment is amplified to produce an amplicon, corresponding to the target DNA sequence. Typical PCR reactions include 30 or more cycles of denaturation, annealing and elongation. In many cases, the annealing and elongation steps can be performed concurrently, in which case the cycle contains only two steps.

Suitable amplification methods include, but are not limited to PCR (Innis et al., 1990), ligase chain reaction (LCR) (see Wu and Wallace, 1989; Landegren et al., 1988 and Barringer et al., 1990), transcription amplification (Kwoh et al., 1989), and self-sustained sequence replication (Guatelli, et al. 1990).

1. Primers

In certain aspects, each primer is sufficiently long to prime the template-directed synthesis of the target nucleic acid sequence under the conditions of the amplification reaction. In certain embodiments, the lengths of the primers depends on many factors, including, but not limited to, the desired hybridization temperature between the primers, the target nucleic acid sequence and the complexity of the different target nucleic acid sequences to be amplified, and other factors. In certain embodiments, a primer is about 15 to about 35 nucleotides in length. In certain embodiments, a primer is fewer than 15 nucleotides in length. In certain embodiments, a primer is greater than 35 nucleotides in length.

In a further aspect, a forward primer can comprise at least one sequence that anneals to a target RNA and alternatively can comprise an additional 5′ non-complementary region, which may be designed to provide an appropriate annealing profile. The forward primer sequence will be dictated in part by the target RNA.

In still a further aspect, a reverse primer is designed to anneal to the complement of a reverse transcribed RNA. In certain aspects of the invention, the reverse primer sequence is generally independent of the target RNA and/or probe segment as is determined by the RT primer. Multiple target RNAs may be amplified using the same reverse primer, e.g., a universal reverse primer. Typically, the characteristics of the forward and reverse primers are compatible and result in an amplification product at the appropriate temperatures.

2. Probes and Labels

In certain embodiments, a probe may include Watson-Crick bases or modified bases. Modified bases include, but are not limited to, the AEGIS bases (from Eragen Biosciences), which have been described, e.g., in U.S. Pat. Nos. 5,432,272; 5,965,364; and 6,001,983. In certain aspects, bases are joined by a natural phosphodiester bond or a different chemical linkage. Different chemical linkages include, but are not limited to, a peptide bond or an LNA linkage, which is described, e.g., in published PCT applications WO 00/56748 and WO 00/66604.

In a further aspect, oligonucleotide probes present in a multiplex amplification are suitable for monitoring the amount of amplification product produced as a function of time. Such oligonucleotide probes include, but are not limited to, the 5′-exonuclease assay (e.g., TaqMan™) probes (see above and also U.S. Pat. No. 5,538,848), stem-loop molecular beacons (see, e.g., U.S. Pat. Nos. 6,103,476 and 5,925,517 and Tyagi & Kramer, 1996), stemless or linear beacons (see, e.g., WO 99/21881), PNA Molecular Beacons (see, e.g., U.S. Pat. Nos. 6,355,421 and 6,593,091), linear PNA beacons (see, e.g. Kubista et al., 2001), non-FRET probes (see, e.g., U.S. Pat. No. 6,150,097), Sunrise®™/Amplifluor®™ probes (see, e.g., U.S. Pat. No. 6,548,250), stem-loop and duplex Scorpion™ probes (see, e.g., Solinas et al., 2001 and U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse™ probe (Epoch Biosciences), hairpin probes (see, e.g., U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001; Whitcombe et al., 1999; Isacsson et al., 2000; Svanvik et al., 2000; Wolffs et al., 2001; Tsourkas et al., 2002; Riccelli et al., 2002; Zhang et al., 2002; Maxwell et al., 2002; Broude et al., 2002; Huang et al., 2002; and Yu et al., 2001.

In certain aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody/antigen, ionic complexes, hapten/ligand (e.g., biotin/avidin). In still other aspects, use of labels can be accomplished using any one of a large number of known techniques employing known labels, linkages, linking groups, reagents, reaction conditions, and analysis and purification methods.

Labels include, but are not limited to, light-emitting, light-scattering, and light-absorbing compounds which generate or quench a detectable fluorescent, chemiluminescent, or bioluminescent signal (see, e.g., Kricka, 1992) and Garman, 1997). Fluorescent reporter dyes useful as labels include, but are not limited to, fluoresceins (see, e.g., U.S. Pat. Nos. 5,188,934; 6,008,379; and 6,020,481), rhodamines (see, e.g., U.S. Pat. Nos. 5,366,860; 5,847,162; 5,936,087; 6,051,719; and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., Kubista, WO 97/45539), as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein. In certain aspects, the fluorescent label is selected from SYBR®-green, 6-carboxyfluorescein (“FAM”), TET, ROX, VIC™, and JOE. In certain embodiments, a label is a radiolabel.

In still a further aspect, labels are hybridization-stabilizing moieties which serve to enhance, stabilize, or influence hybridization of duplexes, e.g., intercalators and intercalating dyes (including, but not limited to, ethidium bromide and SYBR® green), minor-groove binders, and cross-linking functional groups (see, e.g., Blackburn, G. and Gait, M. Eds. “DNA and RNA structure” in Nucleic Acids in Chemistry and Biology (1996). Labels include those labels that effect the separation or immobilization of a molecule by specific or non-specific capture, for example biotin, digoxigenin, and other haptens (see, e.g., Andrus, 1995).

In yet further aspects, different probes comprise detectable and different labels that are distinguishable from one another. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054 and Saiki et al., 1986.

In certain embodiments, one or more of the primers in an amplification reaction can include a label.

3. Polymerases

A polymerase is an enzyme that is capable of catalyzing polymerization of nucleic acids such as RNA and DNA. Numerous diagnostic and scientific applications use polymerases to amplify or synthesize polynucleotides from nucleic acid templates. One application of this method is detecting or isolating nucleic acids present in low copy numbers. In certain aspects, a polymerase is active at 37, 42, 50, 60, 70, 80, 90° C. or higher. In some aspects the polymerase is a thermostable polymerase. Exemplary thermostable polymerases include, but are not limited to, Thermus thermophilus HB8 (see e.g., U.S. Pat. No. 5,789,224 and U.S. publication 20030194726); mutant Thermus oshimai; Thermus scotoductus; Thermus thermophilus 1B21; Thermus thermophilus GK24; Thermus aquaticus polymerase (AmpliTaq® FS or Taq (G46D; F667Y) (see e.g., U.S. Pat. No. 5,614,365), Taq (G46D; F667Y; E6811), and Taq (G46D; F667Y; T664N; R660G); Pyrococcus furiosus polymerase; Thermococcus gorgonarius polymerase; Pyrococcus species GB-D polymerase; Thermococcus sp. (strain 9° N-7) polymerase; Bacillus stearothermophilus polymerase; Tsp polymerase; ThermalAce™ polymerase (Invitrogen); Thermus flavus polymerase; Thermus litoralis polymerase and mutants or variants thereof.

Exemplary non-thermostable polymerases include, but are not limited to DNA polymerase I; mutant DNA polymerase I, including, but not limited to, Klenow fragment and Klenow fragment (3′ to 5′ exonuclease minus); T4 DNA polymerase; mutant T4 DNA polymerase; T7 DNA polymerase; mutant T7 DNA polymerase; phi29 DNA polymerase; and mutant phi29 DNA polymerase.

In certain aspects, a hot start polymerase is used in the amplification reaction. A hot start polymerase is a modified form of a DNA Polymerase that requires thermal activation (see for example U.S. Pat. Nos. 6,403,341 and 7,122,355, hereby incorporated by reference in their entirety). Such a polymerase can be used, for example, to further increase sensitivity, specificity, and yield; and/or to further improve low copy target amplification. Typically, the hot start enzyme is provided in an inactive state. Upon thermal activation the modification or modifier is released, generating active enzyme. A number of hot start polymerases are available from various commercial sources, such as Applied Biosystems; Bio-Rad; eEnzyme LLC; Eppendorf North America; Finnzymes Oy; GeneChoice, Inc.; Invitrogen; Jena Bioscience GmbH; MIDSCI; Minerva Biolabs GmbH; New England Biolabs; Novagen; Promega; QIAGEN; Roche Applied Science; Sigma-Aldrich; Stratagene; Takara Mirus Bio; USB Corp.; Yorkshire Bioscience Ltd; and the like.

In certain embodiments, an amplification reaction comprises a blend of polymerases. In certain such embodiments, at least one polymerase possesses exonuclease activity. In certain embodiments, none of the polymerases in an amplification reaction possess exonuclease activity. Exemplary polymerases that may be used in an amplification reaction include, but are not limited to, phi29 DNA polymerase, Taq polymerase, stoffel fragment, Bst DNA polymerase, E. coli DNA polymerase 1, the Klenow fragment of DNA polymerase 1, the bacteriophage T7 DNA polymerase, the bacteriophage T5 DNA polymerase, and other polymerases known in the art. In certain embodiments, a polymerase is inactive in the reaction composition and is subsequently activated at a given temperature.

C. Detection

Equipment and software are readily available for controlling and monitoring amplicon accumulation in PCR and qRT-PCR according to the fluorescent 5′ nuclease assay and other qPCR/qRT-PCR procedures, including the Smart Cycler, commercially available from Cepheid of Sunnyvale, Calif., the LightCycler® (Roche Diagnositcs), the Mx™ qPCR System (Stratgene) and the ABI Prism 7700 Sequence Detection System (TaqMan), commercially available from Applied Biosystems.

### II. Sample Preparation

The methods of the invention are not limited to any particular method of sample preparation. A large number of well-known methods for isolating and purifying RNA are suitable for this invention.

One of skill in the art will appreciate that it is desirable to have nucleic acid samples containing target nucleic acid sequences that reflect the RNA of interest. Thus, a target DNA reverse transcribed from a RNA, a DNA amplified from the target DNA, etc., are all derived from the RNA target and detection of such derived products is indicative of the presence and/or abundance of the original RNA in a sample. Thus, suitable samples include, but are not limited to, miRNA, siRNA, piwi-interacting RNA, mRNA, and the like. RNA, as used herein, may include, but are not limited to pre-mRNA nascent transcript(s), transcript processing intermediates, mature RNA(s) and degradation products.

In one embodiment, such a sample is a homogenate of cells or tissues or other biological samples. In certain aspects, such sample is a total RNA preparation of a biological sample. In a further aspect, such a sample may be a small RNA preparation of a biological sample.

Biological samples may be of any biological tissue or fluid or cells. Frequently the sample will be a “clinical sample” which is a sample derived from a patient. Clinical samples provide a rich source of information regarding gene expression, a pathological or pre-pathological condition, and/or a diagnostic parameter. Some embodiments of the invention are employed to detect mutations and to identify the function of mutations. Such embodiments have extensive applications in clinical diagnostics and clinical studies. Typical clinical samples include, but are not limited to, sputum, blood, blood cells (e.g., white cells), tissue or fine needle biopsy samples, urine, peritoneal fluid, and pleural fluid, or cells there from. Biological samples may also include sections of tissues such as frozen sections, or sections otherwise preserved or mounted for sectioning and/or histological analysis. In certain aspects, samples are fresh samples or fixed samples, such as formalin or formaldehyde fixed paraffin embedded samples (FFPE).

Another typical source of biological samples are cell cultures where gene expression states can be manipulated to explore the relationship among genes.

One of skill in the art would appreciate that it is desirable to inhibit or destroy RNase present in samples before the samples can be analyzed. Methods of inhibiting or destroying nucleases are well known in the art. In some aspects, cells or tissues are homogenized in the presence of chaotropic agents to inhibit nucleases. In some other aspects, RNase are inhibited or destroyed by heat treatment followed by proteinase treatment.

Methods of isolating RNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993). In a certain aspects, the total RNA is isolated from a given sample using, for example, an acid guanidinium-phenol-chloroform extraction method (see, e.g., Sambrook et al., (1989), or Ausubel et al. (1987). In one aspect, total RNA can be isolated from mammalian cells using RNeasy™ Total RNA isolation kit (QIAGEN). If mammalian tissue is used as the source of RNA, a commercial reagent such as TRIzol™ Reagent (GIBCOL Life Technologies) may be used. A second cleanup after the ethanol precipitation step in the TRIzol™ extraction using Rneasy™ total RNA isolation kit may be beneficial. Hot phenol protocol described by Schmitt et al., (1990) is useful for isolating total RNA for yeast cells.

Total RNA from prokaryotes, such as E. coli cells, may be obtained by following the protocol for MasterPure™ complete DNA/RNA purification kit from Epicentre Technologies (Madison, Wis.) or for RiboPure™ Bacteria kit (Ambion).

### III. Diagnostics

Embodiments of the invention include methods for diagnosing and/or assessing a condition or potential condition in a patient comprising measuring expression of one or more RNA, such as a miRNA, in a sample from a patient. The difference in the expression in the sample from a patient and a reference, such as expression in a normal or non-pathologic sample, is indicative of a pathologic, disease, or cancerous condition, or risk thereof. A sample may be taken from a patient having or suspected of having a disease or pathological condition. In certain aspects, the sample can be, but is not limited to tissue (e.g., biopsy, particularly fine needle biopsy), blood, serum, plasma, or a pancreatic juice samples. The sample can be fresh, frozen, fixed (e.g., formalin fixed), or embedded (e.g., paraffin embedded).

The present invention is of particular interest in the diagnostic screening of RNA samples for many diseases or conditions. In certain embodiments, diagnostic methods involve identifying one or more RNA, such as miRNAs or mRNAs, differentially expressed in a sample that are indicative of a disease or condition (non-normal sample). In certain embodiments, diagnosing a disease or condition involves detecting and/or quantifying an expressed miRNA or mRNA. RNAs clearly linked to a disease phenotype are referred to as “biomarkers.” In certain embodiments, the invention provides for the detection of amplicons that are shorter (<25 nt) than by traditional qRTPCR methods that rely on longer amplicons (60-200 nt). Clinical samples are often subject to extensive RNA degradation, which can limit the sensitivity of detection if the amplicon size of the target is approximately the same size as the degraded RNA or larger. The use of shorter amplicons to detect the target improves the likelihood that the target will exponentially amplified even if highly degraded. Moreover, the use of shorter target-specific amplicons to detect RNA can offer sensitive quantification of RNA in FFPE samples, where the RNA can be compromised by covalently modifications through the fixation process, as well as degraded by the high temperatures used in the embedding process.

The invention may also be used for the detection of RNA in infectious disease, such as RNA viruses such as HIV, HCV, and other microbes. The invention may also have utility for the detection of disease specific RNA (e.g., fusion transcripts) in diseases such as leukemia, where the knowledge of the precise molecular translocation can have prognostic value, as well as guide therapeutic decision-making and other aspects of disease management.

Particularly the methods can be used to evaluate samples with respect to diseases or conditions that include, but are not limited to: Alzheimer's disease, macular degeneration, chronic pancreatitis; pancreatic cancer; AIDS, autoimmune diseases (rheumatoid arthritis, multiple sclerosis, diabetes—insulin-dependent and non-independent, systemic lupus erythematosus and Graves disease); cancer (e.g., malignant, benign, metastatic, precancer); cardiovascular diseases (heart disease or coronary artery disease, stroke-ischemic and hemorrhagic, and rheumatic heart disease); diseases of the nervous system; and infection by pathogenic microorganisms (Athlete's Foot, Chickenpox, Common cold, Diarrheal diseases, Flu, Genital herpes, Malaria, Meningitis, Pneumonia, Sinusitis, Skin diseases, Strep throat, Tuberculosis, Urinary tract infections, Vaginal infections, Viral hepatitis); inflammation (allergy, asthma); prion diseases (e.g., CJD, kuru, GSS, FFI).

Cancers that may be evaluated by methods and compositions of the invention include cancer cells that include cells and cancer cells from the bladder, blood, bone, bone marrow, brain, breast, colon, esophagus, gastrointestine, gum, head, kidney, liver, lung, nasopharynx, neck, ovary, pancreas, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Moreover, RNA can be evaluated in pre-cancers, such as metaplasia, dysplasia, and hyperplasia.

It is specifically contemplated that the invention can be used to evaluate differences between stages of disease, such as between hyperplasia, neoplasia, pre-cancer and cancer, or between a primary tumor and a metastasized tumor.

Moreover, it is contemplated that samples that have differences in the activity of certain pathways may also be compared. These pathways include the following and those involving the following factors: antibody response, apoptosis, calcium/NFAT signaling, cell cycle, cell migration, cell adhesion, cell division, cytokines and cytokine receptors, drug metabolism, growth factors and growth factor receptors, inflammatory response, insulin signaling, NFκ-B signaling, angiogenesis, adipogenesis, cell adhesion, viral infecton, bacterial infection, senescence, motility, glucose transport, stress response, oxidation, aging, telomere extension, telomere shortening, neural transmission, blood clotting, stem cell differentiation, G-Protein Coupled Receptor (GPCR) signaling, and p53 activation.

Cellular pathways that may be assessed also include, but are not limited to, the following: an adhesion or motility pathway including but not limited to those involving cyclic AMP, protein kinase A, G-protein couple receptors, adenylyl cyclase, L-selectin, E-selectin, PECAM, VCAM-1, α-actinin, paxillin, cadherins, AKT, integrin-α, integrin-β, RAF-1, ERK, PI-3 kinase, vinculin, matrix metalloproteinases, Rho GTPases, p85, trefoil factors, profilin, FAK, MAP kinase, Ras, caveolin, calpain-1, calpain-2, epidermal growth factor receptor, ICAM-1, ICAM-2, cofilin, actin, gelsolin, RhoA, RAC 1, myosin light chain kinase, platelet-derived growth factor receptor or ezrin; any apoptosis pathway including, but not limited to, those involving AKT, Fas ligand, NFκB, caspase-9, PI3 kinase, caspase-3, caspase-7, ICAD, CAD, EndoG, Granzyme B, Bad, Bax, Bid, Bak, APAF-1, cytochrome C, p53, ATM, Bcl-2, PARP, Chk1, Chk2, p21, c-Jun, p73, Rad51, Mdm2, Rad50, c-Abl, BRCA-1, perforin, caspase-4, caspase-8, caspase-6, caspase-1, caspase-2, caspase-10, Rho, Jun kinase, Jun kinase kinase, R1p2, lamin-A, lamin-B1, lamin-B2, Fas receptor, H2O2, Granzyme A, NADPH oxidase, HMG2, CD4, CD28, CD3, TRADD, IKK, FADD, GADD45, DR3 death receptor, DR4/5 death receptor, FLIPs, APO-3, GRB2, SHC, ERK, MEK, RAF-1, cyclic AMP, protein kinase A, E2F, retinoblastoma protein, Smac/Diablo, ACH receptor, 14-3-3, FAK, SODD, TNF receptor, RIP, cyclin-D1, PCNA, Bcl-XL, PIP2, PIP3, PTEN, ATM, Cdc2, protein kinase C, calcineurin, IKKα, IKKβ, IKKγ, SOS-1, c-FOS, Traf-1, Traf-2, IκBβ or the proteasome; any cell activation pathway including, but not limited to, those involving protein kinase A, nitric oxide, caveolin-1, actin, calcium, protein kinase C, Cdc2, cyclin B, Cdc25, GRB2, SRC protein kinase, ADP-ribosylation factors (ARFs), phospholipase D, AKAP95, p68, Aurora B, CDK1, Eg7, histone H3, PKAc, CD80, PI3 kinase, WASP, Arp2, Arp3, p16, p34, p20, PP2A, angiotensin, angiotensin-converting enzyme, protease-activated receptor-1, protease-activated receptor-4, Ras, RAF-1, PLCβ, PLCγ, COX-1, G-protein-coupled receptors, phospholipase A2, IP3, SUMO1, SUMO 2/3, ubiquitin, Ran, Ran-GAP, Ran-GEF, p53, glucocorticoids, glucocorticoid receptor, components of the SWI/SNF complex, RanBP1, RanBP2, importins, exportins, RCC1, CD40, CD40 ligand, p38, IKKα, IKKβ, NFκB, TRAF2, TRAF3, TRAF5, TRAF6, IL-4, IL-4 receptor, CDK5, AP-1 transcription factor, CD45, CD4, T cell receptors, MAP kinase, nerve growth factor, nerve growth factor receptor, c-Jun, c-Fos, Jun kinase, GRB2, SOS-1, ERK-1, ERK, JAK2, STAT4, IL-12, IL-12 receptor, nitric oxide synthase, TYK2, IFNγ, elastase, IL-8, epithelins, IL-2, IL-2 receptor, CD28, SMAD3, SMAD4, TGFβ or TGFβ receptor; any cell cycle regulation, signaling or differentiation pathway including but not limited to those involving TNFs, SRC protein kinase, Cdc2, cyclin B, Grb2, Sos-1, SHC, p68, Aurora kinases, protein kinase A, protein kinase C, Eg7, p53, cyclins, cyclin-dependent kinases, neural growth factor, epidermal growth factor, retinoblastoma protein, ATF-2, ATM, ATR, AKT, CHK1, CHK2, 14-3-3, WEE1, CDC25 CDC6, Origin Recognition Complex proteins, p15, p16, p27, p21, ABL, c-ABL, SMADs, ubiquitin, SUMO, heat shock proteins, Wnt, GSK-3, angiotensin, p73 any PPAR, TGFα, TGFβ, p300, MDM2, GADD45, Notch, cdc34, BRCA-1, BRCA-2, SKP1, the proteasome, CUL1, E2F, p107, steroid hormones, steroid hormone receptors, IκBα, IκBβ, Sin3A, heat shock proteins, Ras, Rho, ERKs, IKKs, PI3 kinase, Bcl-2, Bax, PCNA, MAP kinases, dynein, RhoA, PKAc, cyclin AMP, FAK, PIP2, PIP3, integrins, thrombopoietin, Fas, Fas ligand, PLK3, MEKs, JAKs, STATs, acetylcholine, paxillin calcineurin, p38, importins, exportins, Ran, Rad50, Rad51, DNA polymerase, RNA polymerase, Ran-GAP, Ran-GEF, NuMA, Tpx2, RCC1, Sonic Hedgehog, Crml, Patched (Ptc-1), MPF, CaM kinases, tubulin, actin, kinetochore-associated proteins, centromere-binding proteins, telomerase, TERT, PP2A, c-MYC, insulin, T cell receptors, B cell receptors, CBP, IKβ, NFκB, RAC1, RAF1, EPO, diacylglycerol, c-Jun, c-Fos, Jun kinase, hypoxia-inducible factors, GATA4, β-catenin, α-catenin, calcium, arrestin, survivin, caspases, procaspases, CREB, CREM, cadherins, PECAMs, corticosteroids, colony-stimulating factors, calpains, adenylyl cyclase, growth factors, nitric oxide, transmembrane receptors, retinoids, G-proteins, ion channels, transcriptional activators, transcriptional coactivators, transcriptional repressors, interleukins, vitamins, interferons, transcriptional corepressors, the nuclear pore, nitrogen, toxins, proteolysis, or phosphorylation; any metabolic pathway including but not limited to those involving the biosynthesis of amino acids, oxidation of fatty acids, biosynthesis of neurotransmitters and other cell signaling molecules, biosynthesis of polyamines, biosynthesis of lipids and sphingolipids, catabolism of amino acids and nutrients, nucleotide synthesis, eicosanoids, electron transport reactions, ER-associated degradation, glycolysis, fibrinolysis, formation of ketone bodies, formation of phagosomes, cholesterol metabolism, regulation of food intake, energy homeostasis, prothrombin activation, synthesis of lactose and other sugars, multi-drug resistance, biosynthesis of phosphatidylcholine, the proteasome, amyloid precursor protein, Rab GTPases, starch synthesis, glycosylation, synthesis of phoshoglycerides, vitamins, the citric acid cycle, IGF-1 receptor, the urea cycle, vesicular transport, or salvage pathways. It is further contemplated that nucleic acids molecules of the invention can be employed in diagnostic and therapeutic methods with respect to any of the above pathways or factors. Thus, in some embodiments of the invention, a RNA may be differentially expressed with respect to one or more of the above pathways or factors.

Phenotypic traits also include characteristics such as longevity, morbidity, appearance (e.g., baldness, obesity), strength, speed, endurance, fertility, susceptibility or receptivity to particular drugs or therapeutic treatments (drug efficacy), and risk of drug toxicity. Samples that differ in these phenotypic traits may also be evaluated using the methods described.

In certain embodiments, RNA profiles may be generated to evaluate and correlate those profiles with pharmacokinetics. For example, RNA profiles may be created and evaluated for patient tumor and blood samples prior to the patient's being treated or during treatment to determine if there are RNAs whose expression correlates with the outcome of the patient. Identification of differential RNAs can lead to a diagnostic assay involving them that can be used to evaluate tumor and/or blood samples to determine what drug regimen the patient should be provided. In addition, the methods can be used to identify or select patients suitable for a particular clinical trial. If a RNA profile is determined to be correlated with drug efficacy or drug toxicity, that may be relevant to whether that patient is an appropriate patient for receiving the drug or for a particular dosage of the drug.

In addition to the above prognostic assay, blood samples from patients with a variety of diseases can be evaluated to determine if different diseases can be identified based on blood RNA levels. A diagnostic assay can be created based on the profiles that doctors can use to identify individuals with a disease or who are at risk to develop a disease.

### IV. Kits

Any of the compositions or reagents described herein may be comprised in a kit. In a non-limiting example, reagents for reverse transcribing a RNA target, such as a miRNA, using a RT primer comprising in a 5″ to 3′ direction a primer segment, a probe segment, and a target specific annealing segment are included in a kit. The kit may also include multiple RT primers to multiple sites on one or more RNA. The kit may also comprise reagents for reverse transcribing RNA to a DNA template and/or reagents, including primers, for amplification of the target DNA. Such a kit may include one or more buffers, such as a reaction, amplification, and/or a transcription buffer, compounds for preparing a RNA sample, and components for isolating and/or detecting an amplification product, such as probe or label.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (RT reagent and amplification reagents may be packaged together), the kit also will generally contain a second, third or other additional container into which the additional components may be separately placed. However, various combinations of components may be comprised in one or more vial. The kits of the present invention also will typically include a container for primers and probes, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power.

The container means will generally include at least one vial, test tube, flask, bottle, syringe and/or other container means, into which reactions are placed or allocated and/or reaction methods are performed. The kits may also comprise a second container means for containing a buffer and/or other diluent.

A kit will also include instructions for employing the kit components as well the use of any other reagent not included in the kit. Instructions may include variations that can be implemented.

Kits of the invention may also include one or more of the following in addition to a RT primer: 1) RT buffer; 2) Control RNA template; 3) reverse transcriptase and/or polymerase; 4) RT or polymerase buffer; 5) dNTPs and/or NTPs; 6) nuclease-free water; and/or 7) RNase-free containers, such as 1.5 ml tubes, as well as other reagents.

It is contemplated that such reagents are embodiments of kits of the invention. Such kits, however, are not limited to the particular items identified above and may include any labeling reagent or reagent that promotes or facilitates the labeling of a nucleic acid.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

Abbreviations and definitions used in the Examples include the following: RT, reverse transcription; FW, forward; R, reverse; NF, nuclease free; Ct, cycle threshold value—the number of cycles at which the fluorescence of the reporter dye first substantially exceeds the calculated background level; qPCR, quantitative polymerase chain reaction; nt, nucleotide(s); and NTC, no target control.

### Example 1

### Methods for Quantification of RNA

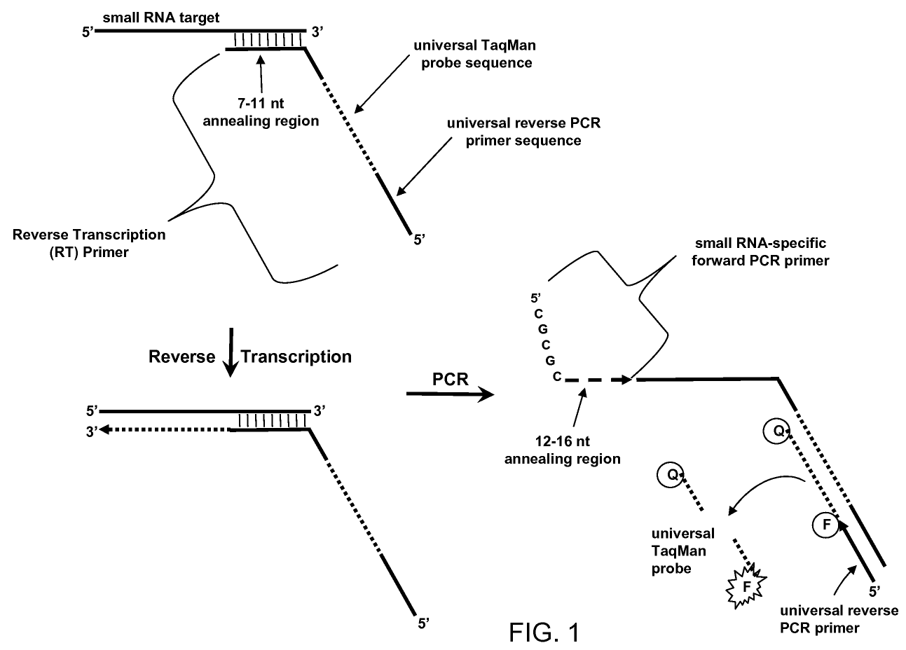

Unless otherwise stated, the examples of the invention employed the methods shown in FIG. 1 and described below for quantification of RNAs. For reverse transcription, reaction components were assembled on ice prior to the addition of the RNA template and are shown in Table 1 below

TABLE 1
Reverse transcription reaction components.

| | μl per | μl per | Final |
|---|---|---|---|
| Component | 10 μl rxn | 15 μl rxn | Concentration |
| nuclease free water | 3.80 | 5.70 | |
| 10X RT buffer | 1.00 | 1.50 | 1X |
| (Retroscipt, Ambion) | | | |
| dNTP mix (2.5 mM each) | 1.00 | 1.50 | 1 mM |
| (Ambion) | | | |
| RT Primer (250 nM) | 2.00 | 3.00 | 50 nM |
| RNase Inhibitor (40 U/μl) | 0.10 | 0.15 | 0.4 U/μl |
| (Ambion) | | | |
| MMLV-RT (100 U/μl) | 0.10 | 0.15 | 1 U/μl |
| (Ambion) | | | |
| RNA template | 2.00 | 3.00 | |

Following assembly of the reaction components on ice, the RNA template (1 pg-40 ng) was added to the reaction mix. If a RNA template was a synthetic RNA, it was added in a background of 10 ng/μl of polyA RNA. The reverse transcription reaction was incubated at 16° C. for 15 min, then at 42° C. for 15 to 30 min, then at 85° C. to 95° C. for 5 to 10 min.

For the PCR, the reaction components shown below (Table 2) are assembled on ice, prior to the addition of the cDNA from the reverse transcriptase reaction above.

TABLE 2
qPCR components.

| | μl per | μl per | Final |
|---|---|---|---|
| Component | 10 μl rxn | 15 μl rxn | Concentration |
| nuclease free water | 3.53 | 5.30 | |
| MgCl₂ (50 mM) | 1.00 | 1.50 | 5 mM |
| 10X Platinum Taq PCR buffer | 1.00 | 1.50 | 1X |
| dNTP mix (2.5 mM each) | 1.00 | 1.50 | 1 mM |
| PCR universal TaqMan probe | 0.40 | 0.60 | 80 nM |
| (2 μM) | | | |
| PCR forward primer (10 μM) | 0.30 | 0.45 | 0.3 μM |
| PCR universal reverse primer | 0.50 | 0.75 | 0.5 μM |
| (10 μM) | | | |
| 50X ROX internal marker | 0.20 | 0.30 | 1X |
| Platinum Taq polymerase | 0.07 | 0.10 | 0.033 U/μl |
| (5 U/μl) | | | |
| cDNA from RT reaction | 2.00 | 3.00 | |

Following assembly of the reaction components on ice, 1/5 volume of the corresponding reverse transcription reaction was transferred to the PCR mix. PCRs were incubated at 95° C. for 1 min, then for 40 to 50 cycles of 95° C. for 3 to 15 sec and 60° C. for 30 to 45 sec.

### Example 2

### Quantification of RNA Using a Taqman™ Probe

This experiment demonstrates the use of a universal TaqMan probe in qPCR defined by the reverse transcription (RT) primer to detect small, as well as large, RNA. Variously sized fragments of the human apolipoprotein E mRNA (apoE; GenBank accession number NM_{—}000041, incorporated herein by reference) were amplified from total human liver RNA (Ambion) using the primers shown in Table 3 and the reaction conditions described below. For the apoE RT primer, only six nucleotides at the 3′ end (CTGCAT) are complementary to and mediate binding to the apoE mRNA.

TABLE 3

PCR Primers and amplicon sizes for amplifica-
tion of apoE mRNA.

| | | | |
|---|---|---|---|
| | | | Ampli- |
| | | | con |
| Gene | Primer | Sequence | Size |

| | | | |
|---|---|---|---|
| apoE | apoE | 5′- | |
| | RT | GGTCCGACTACCCCAACAATACCTTGAACC | |
| | Primer | CTACAGCAGAGTCTGCAT-3′ | |
| | | (SEQ ID NO: 1) | |
| apoE | PCR | 5′-GCGCCCTGGTGGAAGACA-3′ | 20 |
| | FW-20 | (SEQ ID NO: 2) | |
| apoE | PCR | 5′-GCTTCCAGGCCCGCC-3′ | 52 |
| | FW-52 | (SEQ ID NO: 3) | |
| apoE | PCR | 5′-GCCAAGCTGGAGGAGCAG-3′ | 99 |
| | FW-99 | (SEQ ID NO: 4) | |
| apoE | PCR | 5′-GGCAGTGTACCAGGCCG-3′ | 343 |
| | FW-343 | (SEQ ID NO: 5) | |
| apoE | PCR | 5′-GGAGCCGACTGGCCAAT-3′ | 903 |
| | FW-903 | (SEQ ID NO: 6) | |
| Uni- | PCR | 5′-GGTCCGACTACCCCAACAA-3 | |
| versal | uni- | (SEQ ID NO: 7) | |
| | versal | | |
| | re- | | |
| | verse | | |
| Uni- | PCR | 5′-6FAM-CTTGAACCCTACAGCAGAGT- | |
| versal | uni- | MGBNFQ-3′ | |
| | versal | (SEQ ID NO: 8) | |
| | TaqMan | | |
| | probe | | |

Two-Step RT-PCR—Reverse transcription reaction mixtures (15 μl) contained 3.7 μl of nuclease free water, 1.5 μl of 10×RT buffer (Retroscript, Ambion), 1.5 μl of dNTP mix (2.5 mM each dNTP), 0.15 μl of Ribonuclease Inhibitor Protein (40 U/μl; Ambion), 0.15 μl MMLV RT (100 U/μl), 5 μl (0.5 ng, 5 ng or 500 ng) of human liver total RNA (Ambion, cat. no. 7960), and 3 μl of apoe RT primer (250 nM). The reactions were incubated at 16° C. for 30 min, then at 42° C. for 30 min, and finally at 95° C. for 10 min in a thermocycler (GeneAmp PCR system 9700, Applied Biosystems).

For qPCR, one third (5 μl) of each RT reaction was transferred into 15 μl qPCRs. qPCRs were individually prepared with one of five apoE forward primers (Table 3) designed to amplify mRNA fragments of five different lengths. Each reaction contained a PCR FW primer (0.87 μM final), PCR universal reverse primer (0.47 μM final), the PCR universal TaqMan probe (67 nM final), and SuperTaq (Ambion, cat. no. 2052) at 0.04 U/μL (final). qPCRs were incubated at 95° C. for 5 min, then for 40 cycles at 95° C. for 15 sec and 60° C. for 1 min. Incubations were carried out in a 7900HT Fast Real-Time PCR System. Real-time qPCR data were analyzed with the SDS 2.3 program (Applied Biosystems). The results of the analyses are shown in Table 4.

TABLE 4
Average Ct of duplicate qPCR for apoE amplicons of
various sizes amplified from various amounts of total
human liver RNA.

| | |
|---|---|
| apoE | Total RNA in |
| Amplicon | Reverse Transcription Reaction |

| Size | 500 ng | 5 ng | 0.5 ng | 0 ng (NTC) |
|---|---|---|---|---|
| 20 nt | 23.89 | 27.95 | 31.05 | 36.47 |
| 52 nt | 26.67 | 29.18 | 32.70 | 35.00 |
| 99 nt | 22.50 | 25.40 | 26.56 | 26.94 |
| 343 nt | 25.83 | 30.17 | 32.30 | 35.97 |
| 903 nt | 26.90 | 31.18 | 30.98 | 32.17 |

The results demonstrate that the methods of the invention can specifically detect the target RNA in as little as 500 pg of total RNA and in as much as 500 ng of total RNA. Amplicons as small as 20 nt and as large as 900 nt are readily detected and quantified.

### Example 3

### Quantification of RNA Using a Molecular Beacon Probe

This study demonstrates the use of a molecular beacon probe, defined by the RT primer, in qPCR to detect small RNAs. The RT reaction and qPCR were assembled as described in Example 1 using a 15 μl volume for each reaction, except that a molecular beacon probe (Beacon165 HCV-3a; 5′-6FAM-CACCGTTAGTACGAGTGTCGGTG-BHQ1-3′ SEQ ID NO:9) replaced the TaqMan™ probe in the reaction. The RT and qPCR incubation conditions were modified for use with a molecular beacon. The RT was carried out at 48° C. for 30 min followed by 95° C. for 5 min. qPCRs were incubated at 95° C. for 5 min, then for 50 cycles of 95° C. for 5 min, 53° C. for 1 min, and 72° C. for 30 sec. Template RNA in the RT reaction included 1,000, 5,000, or 25,000 copies of synthetic hsa-miR-143 or synthetic hsa-miR-205. For hsa-miR-143, the RT primer was RT 8 (5′-GGTCCGACTACCCCAACAATACCACCGTTAG TACGAGTGTCGGTGTGAGCTAC-3′ SEQ ID NO:10) and the PCR forward primer was FW 13 (5′-CGCGCCTGAGATGAAGCAC-3′ SEQ ID NO:11). For hsa-miR-205, the RT primer was RT 9 (5′-GGTCCGACTACCCCAACAATACCACCGTTAGTACGAGTGTCGGTGC AGACTCCG-3′ SEQ ID NO:12) and the PCR forward primer was FW 12 (5′-CGCGCCTCCTTCATTCCA-3′ SEQ ID NO:13). The results are shown in Table 5.

TABLE 5
Average Ct from duplicate qPCRs containing various copy
numbers of hsa-miR-143 and hsa-miR-205 detected with
a molecular beacon.

| | Target RNA Copies | | | | | | | | |
|---|---|---|---|---|---|---|---|---|---|
| Target | 0 (NTC) | | 1,000 | | 5,000 | | 25,000 | | |
| RNA | Ct | SD | Ct | SD | Ct | SD | Ct | SD | Slope |
| hsa- | 36.67 | 1.84 | 33.56 | 0.29 | 31.33 | 0.47 | 28.98 | 0.01 | −3.27 |
| miR- | | | | | | | | | |
| 143 | | | | | | | | | |
| hsa- | 36.26 | 0.70 | 33.61 | 0.24 | 31.41 | 0.19 | 29.33 | 0.10 | −3.06 |
| miR- | | | | | | | | | |
| 205 | | | | | | | | | |

SD, standard deviation.

The results demonstrate the detection of as few as 1,000 copies of synthetic hsa-miR-143 and hsa-miR-205 using a molecular beacon probe for qPCR detection and quantification. Thus, demonstrating that reporter probe technologies other than just TaqMan probes are suitable for use with this invention.

### Example 4

### Specificity of Target Amplification

This study demonstrates improved specificity of target amplification when an RT primer containing ten nucleotides that are complementary to the target RNA is compared with an RT primer containing six nucleotides that are complementary to the target RNA. Here, the amplification products from end-point PCRs were analyzed by agarose gel electrophoresis.

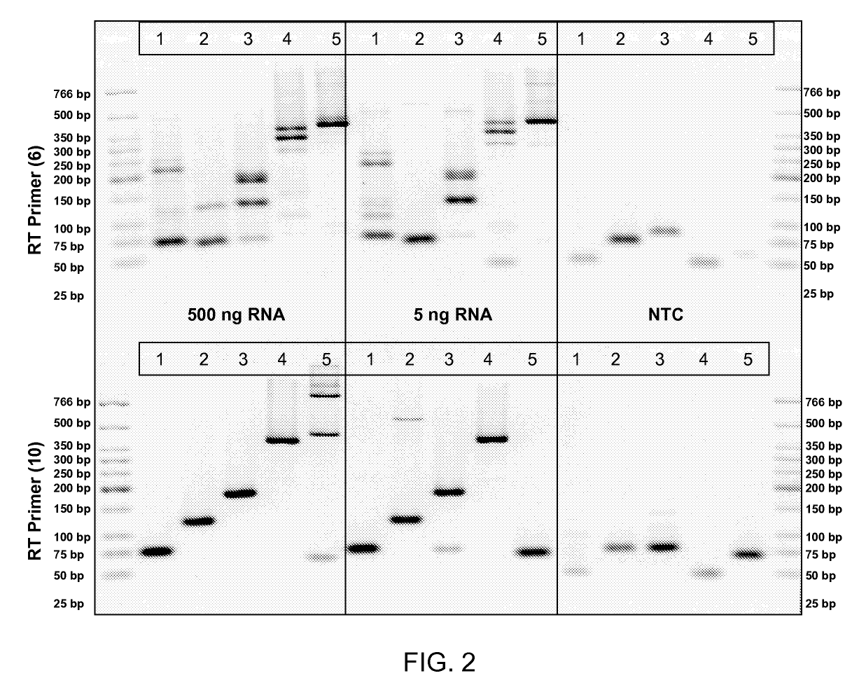

Two-Step End-Point RT-PCR—Reverse transcription reactions were prepared as described in Example 2, except that only two different amounts of human liver total RNA were used (5 ng or 500 ng). One sixth (2.5 μL) of each reverse transcription reaction were used for the end-point PCR. PCRs were individually prepared with one of five FW primers (Table 6) for the human 24-dehydrocholesterol reductase mRNA (DHCR24; Genbank accession number NM_{—}014762, which is incorporated herein by reference). Each reaction contained one FW primer (0.67 μM final), the universal reverse primer (0.4 μM final), and Platinum Taq (Invitrogen, cat. no. 10966-018) (0.5 Upper reaction). PCRs were incubated at 95° C. for 2 min, then for 40 cycles at 95° C. for 30 sec and 60° C. for 30 sec, then for one cycle at 72° C. for 1 min. Incubations were carried out in a GeneAmp PCR system 9700 thermocycler (Applied Biosystems). Reaction products (10 μl of each reaction) were separated on a 2% agarose gel and visualized with SYBR Gold using an AlphaImmager EC (Alpha Innotech Corp.) (FIG. 2).

TABLE 6

PCR Primers and amplicon sizes for amplifica-
tion of DHCR24 mRNA.

| | | | |
|---|---|---|---|
| | | | Am- |
| | | | pli- |
| | | | con |
| Gene | Primer | Sequence | Size |

| | | | |
|---|---|---|---|
| DHCR | DHCR24 | 5′- | |
| 24 | RT | GGTCCGACTACCCCAACAATACCTTGAACCCTA | |
| | Primer | CAGCAGAGTCACATG-3′ | |
| | (6) | (SEQ ID NO: 14) | |
| DHCR | DHCR24 | 5′- | |
| 24 | RT | GGTCCGACTACCCCAACAATACCTTGAACCCTA | |
| | Primer | CAGCAGAGTTTCCACATGC-3′ | |
| | (10) | (SEQ ID NO: 15) | |
| DHCR | PCR | 5′-CCTGATGCCAGTGGGCAGT-3 | 26 |
| 24 | FW-26 | (SEQ ID NO: 16) | |
| DHCR | PCR | 5′-TCCCTGGCCATCCATGTG-3′ | 65 |
| 24 | FW-65 | (SEQ ID NO: 17) | |
| DHCR | PCR | 5′-GGAAAGAGGCAGTCTTCTTAGCAT-3′ | 129 |
| 24 | FW-129 | (SEQ ID NO: 18) | |
| DHCR | PCR | 5′-CGAGCTAGTTAAACAGTGCCATTG-3 | 331 |
| 24 | FW-331 | (SEQ ID NO: 19) | |
| DHCR | PCR | 5′-GGAAAGCCTCATTTGTGTGGAA-3 | 789 |
| 24 | FW-789 | (SEQ ID NO: 20) | |
| Uni- | PCR | 5′-GGTCCGACTACCCCAACAA-3 | |
| ver- | Uni- | (SEQ ID NO: 21) | |
| sal | versal | | |
| | re- | | |
| | verse | | |

The results shown in FIG. 2 demonstrate improved specificity of target detection when an RT primer is used that has a longer region of complementarity with the target RNA (10 nt vs. 6 nt). This effect is observed for amplicons of various sizes.

### Example 5

### Optimization of Primer Length

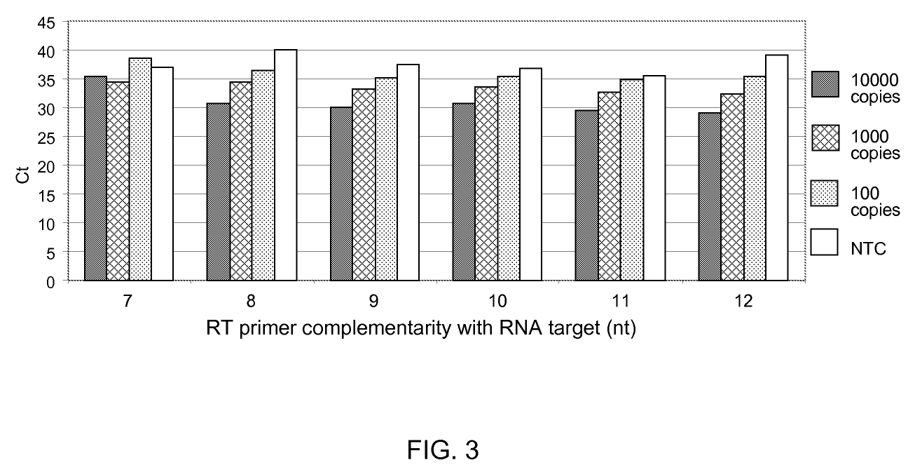

These studies were performed to further evaluate the effect of length of the target complementary region in the RT primer. Six different RT primers (RT7-RT12) having 7-12 bases of complementarity with a target RNA were evaluated. Synthetic hsa-miR-143 (100, 1,000, or 10,000 copies) served as the target RNA in 15 μL reverse transcription reactions. Control reactions with each RT primer and no target RNA (NTC) were also prepared. Methods were as described as in Example 1 except that reverse transcriptions were incubated at 16° C. for 30 min, then at 42° C. for 30 min, then at 95° C. for 10 min in a GeneAmp PCR system 9700 thermocycler (Applied Biosystems). One sixth (2.5 μl) of the RT reaction was transferred into a 15 μL qPCR. All qPCRs employed an identical forward primer (FW 13) at 0.87 μM, the universal reverse primer at 0.47 μM, and Platinum Taq (0.04 U/μl). The universal TaqMan probe was present at a final concentration of 67 nM. The PCR conditions for this experiment consisted of an initial incubation at 95° C. for 5 min, followed by 40 cycles of 95° C. for 15 sec and 60° C. for 1 min. qPCRs were performed in a 7900HT Fast Real-Time PCR System. Real-time data were analyzed using the SDS 2.3 program (Applied Biosystems). Results are shown in Table 7 and in FIG. 3.

TABLE 7
Ct values from duplicate qPCRs, following reverse
transcription with RT primers having different lengths
of complementarity with target RNA.

| RT Primer | Copies of hsa-miR-143 | | | |
|---|---|---|---|---|
| Complementarity | in Reverse Transcription | | | |
| with has-miR-143 (nt) | 10,000 | 1,000 | 100 | 0 (NTC) |
| 7 | 35.32 | 34.54 | 38.64 | 37.00 |
| 8 | 30.63 | 34.48 | 36.37 | 40.00 |
| 9 | 30.06 | 33.27 | 35.12 | 37.43 |
| 10 | 30.79 | 33.52 | 35.39 | 36.84 |
| 11 | 29.45 | 32.69 | 34.8 | 35.59 |
| 12 | 29.13 | 32.34 | 35.44 | 39.13 |

The results indicate that use of an RT primer with seven nucleotides complementary to the target RNA results in poor sensitivity and specificity of RNA detection. In contrast, using RT primers with 8 to 12 complementary nucleotides results in greater sensitivity for detection of the target without an appreciable loss in specificity.

### Example 6

### Optimization of qPCR Forward Primer Length

These studies provide an additional example of optimization of the RT primer length. In addition, these studies evaluate the optimum length of complementarity between the qPCR forward primer and the cDNA target. Seven different RT primers (RT6-RT12) having 6-12 bases of complementarity with a target RNA were evaluated in the method of the invention. Synthetic hsa-miR-375 (100, 1,000, or 10,000 copies) served as the target RNA. RT reactions and qPCRs were performed as described in Example 5, using seven different RT primers (RT6-RT12) designed for use with hsa-miR-375. For the NTC reactions, nuclease free water was added to the reverse transcription in place of hsa-miR-375. qPCRs were performed as in Example 5, except that three different forward primers (FW 12, FW 14, FW 16) with various amounts of complementarity to the cDNA target were used. qPCR data were analyzed as in Example 5 and are shown in Table 8.

The results demonstrate that use of shorter RT primers, in certain embodiments of the invention, results in poorer sensitivity of RNA detection as indicated by higher Ct values in qPCR with 100 copies of target RNA (e.g., RT6). The use of longer RT primers, in combination with forward primers with longer complementary regions, may result in reduced specificity of detection (e.g., RT12 and FW14 or FW16) as indicated by small ACts and lower no target Ct values. In certain embodiments of the invention, the optimal RT primer length will include about 8-10 bases of complementarity with the target RNA sequence and the optimal qPCR FW primer length will include about 12-14 bases of complementarity with the target cDNA sequence.

TABLE 8
Ct values from duplicate qPCRs performed with three different forward primers,
following reverse transcription with RT primers having different
lengths of complementarity with target RNA.

| | | Copies of hsa-miR-375 in Reverse | | | | | | |
|---|---|---|---|---|---|---|---|---|
| | | Transcription | | | | | | |
| RT Primer | | | ΔCt | | ΔCt | | ΔCt | |
| Complementarity with | FW | | vs | | vs | | vs | 0 |
| has-miR-375 (nt) | Primer | 10,000 | NTC | 1,000 | NTC | 100 | NTC | (NTC) |
| 6 | FW 16 | 29.55 | 8.6 | 30.90 | 7.3 | 34.62 | 3.6 | 38.19 |
| | FW 14 | 29.32 | 10.3 | 30.60 | 9.0 | 35.40 | 4.2 | 39.59 |
| | FW 12 | 32.53 | 7.5 | 34.80 | 5.2 | 38.01 | 2.0 | 40.00 |
| 7 | FW 16 | 27.14 | 8.0 | 28.90 | 6.3 | 32.47 | 2.7 | 35.15 |
| | FW 14 | 27.24 | 7.9 | 28.90 | 6.2 | 31.92 | 3.2 | 35.11 |
| | FW 12 | 30.65 | 6.3 | 32.15 | 4.7 | 34.62 | 2.3 | 36.90 |
| 8 | FW 16 | 27.03 | 10.3 | 28.84 | 8.5 | 32.31 | 5.0 | 37.31 |
| | FW 14 | 26.99 | 8.7 | 28.94 | 6.8 | 31.93 | 3.8 | 35.71 |
| | FW 12 | 30.50 | 3.7 | 32.40 | 1.8 | 35.26 | −1.0 | 34.22 |
| 9 | FW 16 | 27.07 | 7.7 | 28.69 | 6.1 | 31.84 | 2.9 | 34.74 |
| | FW 14 | 27.09 | 9.7 | 28.57 | 8.2 | 31.71 | 5.1 | 36.79 |
| | FW 12 | 30.41 | 6.9 | 32.08 | 5.3 | 34.53 | 2.8 | 37.34 |
| 10 | FW 16 | 27.13 | 8.9 | 28.55 | 7.5 | 31.98 | 4.0 | 36.02 |
| | FW 14 | 27.04 | 8.6 | 28.60 | 7.0 | 32.03 | 3.6 | 35.60 |
| | FW 12 | 30.27 | 5.9 | 32.02 | 4.2 | 35.57 | 0.6 | 36.17 |
| 11 | FW 16 | 26.97 | 5.4 | 28.62 | 3.7 | 31.15 | 1.2 | 32.32 |
| | FW 14 | 26.89 | 5.2 | 28.50 | 3.6 | 31.18 | 1.0 | 32.13 |
| | FW 12 | 30.29 | 7.0 | 32.10 | 5.2 | 34.51 | 2.8 | 37.27 |
| 12 | FW 16 | 26.80 | 2.1 | 27.88 | 1.0 | 28.73 | 0.1 | 28.85 |
| | FW 14 | 26.49 | 0.6 | 27.70 | −0.6 | 28.57 | −1.5 | 27.09 |
| | FW 12 | 30.17 | 5.3 | 31.85 | 3.6 | 34.41 | 1.0 | 35.46 |

### Example 7

### 3′ End Complementarity of Primers

When practicing certain embodiments of the invention, e.g., amplification of very short RNAs like miRNAs, the RT primer and the PCR FW primer may have complementary bases at their 3′ ends. For no target control (NTC) reactions, the subsequent transfer of an aliquot of the reverse transcription reaction to the PCR will result in transfer of unused RT primer. This may result in FW primer-RT primer annealing and amplification, leading to a false positive signal that manifests as lower Ct values in the NTC.

RT reactions and qPCRs were performed as described in Example 5, using six different RT primers (RT7-RT12) designed for use with hsa-miR-143. For the NTC reactions, nuclease free water was added to the reverse transcription in place of hsa-miR-143. qPCRs were performed with three different forward primers (FW12, FW13, FW17). qPCR data were analyzed as in Example 5 and are shown in Table 9.

TABLE 9
Ct values from duplicate qPCRs, following reverse transcription
of “no target control” reactions with six RT primers, having
different lengths of complementarity with three PCR FW
primers.

| | PCR | Complementary | |
|---|---|---|---|
| | forward | Nucleotides at | NTC |
| RT Primer | primer | 3′ ends of Primers | Ct |
| RT7 | FW 12 | −3 | 37.5 |
| RT8 | FW 12 | −2 | 40.0 |
| RT7 | FW 13 | −2 | 39.6 |
| RT9 | FW 12 | −1 | 40.0 |
| RT8 | FW 13 | −1 | 40.0 |
| RT10 | FW 12 | 0 | 37.9 |
| RT9 | FW 13 | 0 | 37.1 |
| RT11 | FW 12 | 1 | 40.0 |
| RT10 | FW 13 | 1 | 38.3 |
| RT12 | FW 12 | 2 | 35.3 |
| RT11 | FW 13 | 2 | 40.0 |
| RT7 | FW 17 | 2 | 37.6 |
| RT12 | FW 13 | 3 | 33.4 |
| RT8 | FW 17 | 3 | 34.5 |
| RT9 | FW 17 | 4 | 35.2 |
| RT10 | FW 17 | 5 | 33.6 |
| RT11 | FW 17 | 6 | 27.1 |
| RT12 | FW 17 | 7 | 21.2 |

The results demonstrate that increasing complementarity between the 3′ ends of the RT and PCR FW primers is detrimental to the specificity of the assay. Lower Ct values (≦35) (false positives) may result when the primers have 3 or more complementary nucleotides at their 3′ ends. In contrast, when the primers have 2 or fewer complementary nucleotides at their 3′ ends, the NTC show higher Ct values, generally >35. In certain embodiments of the invention, the optimal complementarity between the RT primer and the qPCR forward primer will be ≦2 nucleotides.

### Example 8

### Enhancing Sensitivity of Detection

The studies demonstrate that, in some embodiments of the invention, the presence of additional non-templated nucleotides (“tails”) on the qPCR forward primer, 5′ of the annealing region, enhances the sensitivity of target detection. The methods of the invention were performed as described in Example 1 using 15 μl RT and qPCR reactions. The sensitivities of detection of 1,000 copies of two synthetic target RNAs were evaluated (hsa-miR-143 and hsa-miR-205). RT primers were miR-143-RT8 or miR-205-RT9. Various sequences were appended 5′ of the annealing regions of the miR-143 and miR-205 qPCR FW primers and evaluated in the methods of the invention. Primers and results are shown in Table 10.

Forward primers miR-143 FW 13-0 and miR-205 FW 12-0 represent the qPCR FW primers with no 5′ tail. Results demonstrate that qPCR FW primers with no tails provide the least sensitive detection in the methods of the invention. Sensitive detection of hsa-miR-143, was achieved with qPCR FW primers having two or more “GC-rich” nucleotides appended to the 5′ end. For sensitive detection of hsa-miR-143, appending “AT-rich” tails to the 5′ end of the FW primer was much less effective. Sensitive detection of hsa-miR-205 was achieved with qPCR FW primers having four or more “GC-rich” nucleotides appended to the 5′ end.

TABLE 10

Optimization of forward primer 5′ tail for detection of synthetic
hsa-miR-143 and hsa-miR-205.

| | | | | | | |
|---|---|---|---|---|---|---|
| | | | Avg | | | |
| Forward | | Tm | Ct | S.D. | Avg | |
| Primer | Sequence 5′–3′ | ° C. | NTC | NTC | Ct | S.D. |

| | | | | | | |
|---|---|---|---|---|---|---|
| miR143 | CGCGCCTGAGATGAAGCAC | 59.5 | 45.07 | 4.84 | 31.2 | 0.10 |
| FW 13-1 | (SEQ ID NO: 22) | | | | | |
| miR143 | GCGCCTGAGATGAAGCAC | 57.0 | 44.50 | 5.67 | 31.14 | 0.32 |
| FW 13-2 | (SEQ ID NO: 23) | | | | | |
| miR143 | CGCCTGAGATGAAGCAC | 53.8 | 44.65 | 7.45 | 31.79 | 0.52 |
| FW 13-3 | (SEQ ID NO: 24) | | | | | |
| miR143 | GCCTGAGATGAAGCAC | 50.4 | 49.49 | 0.88 | 30.52 | 0.16 |
| FW 13-4 | (SEQ ID NO: 25) | | | | | |
| miR143 | CCTGAGATGAAGCAC | 46.2 | 49.34 | 1.14 | 31.90 | 0.26 |
| FW 13-5 | (SEQ ID NO: 26) | | | | | |
| miR143 | CTGAGATGAAGCAC | 42.3 | 50 | 0 | 50 | 0 |
| FW 13-6 | (SEQ ID NO: 27) | | | | | |
| miR143 | GTGAGATGAAGCAC | 42.6 | 47.91 | 3.62 | 39.39 | 1.24 |
| FW 13-7 | (SEQ ID NO: 28) | | | | | |
| miR143 | TGAGATGAAGCAC | 39.6 | 50 | 0 | 50 | 0 |
| FW 13-0 | (SEQ ID NO: 29) | | | | | |
| miR205 | CGCGCCTCCTTCATTCCA | 58.4 | 50 | 0 | 35.91 | 0.49 |
| FW 12-1 | (SEQ ID NO: 30) | | | | | |
| miR205 | GCGCCTCCTTCATTCCA | 55.6 | 50 | 0 | 36.57 | 1.67 |
| FW 12-2 | (SEQ ID NO: 31) | | | | | |
| miR205 | CGCCTCCTTCATTCCA | 51.9 | 48.44 | 2.70 | 35.93 | 0.36 |
| FW 12-3 | (SEQ ID NO: 32) | | | | | |
| miR205 | GCCTCCTTCATTCCA | 48.1 | 50 | 0 | 39.54 | 0.51 |
| FW 12-4 | (SEQ ID NO: 33) | | | | | |
| miR205 | CCTCCTTCATTCCA | 43.2 | 50 | 0 | 50 | 0 |
| FW 12-5 | (SEQ ID NO: 34) | | | | | |
| miR205 | CTCCTTCATTCCA | 38.5 | 50 | 0 | 50 | 0 |
| FW 12-6 | (SEQ ID NO: 35) | | | | | |
| miR205 | TCCTTCATTCCA | 35.1 | 50 | 0 | 50 | 0 |
| FW 12-0 | (SEQ ID NO: 36) | | | | | |
| miR143 | TATGAGATGAAGCAC | 41.1 | 50 | 0 | 50 | 0 |
| FW 13-14 | (SEQ ID NO: 37) | | | | | |
| miR143 | TATATGAGATGAAGCAC | 42.4 | 47.75 | 3.90 | 43.46 | 2.89 |
| FW 13-15 | (SEQ ID NO: 38) | | | | | |
| miR143 | ATATATATATATATGAGATGAAGCAC | 46.3 | 47.67 | 2.63 | 32.61 | 0.23 |
| FW 13-16 | (SEQ ID NO: 39) | | | | | |
| miR143 | ATATATATATATATATATATATATATATAT | 50.5 | 50 | 0 | 42.32 | 0.04 |
| FW 13-17 | ATGAGATGAAGCAC | | | | | |
| | (SEQ ID NO: 40) | | | | | |
| miR143 | ATATATATATATATATATATATATATA | 53.3 | 50 | 0 | 40.05 | 0.29 |
| FW 13-8 | TATATATATATATATATATATGAGATGAAGCAC | | | | | |
| | (SEQ ID NO: 41) | | | | | |

### Example 9

### Evaluation of Rt Primer Concentration

Various concentrations of RT primer were evaluated for the quantification of hsa-miR-16 and hsa-miR-205 using the methods of the invention. RT reactions (10 μl) and qPCRs were performed as described in Example 1, except that 3 μl of each RT reaction was transferred into a 15 μl qPCR. Pooled human total RNA (50 pg) was added to RT reactions and consisted of a mixture of total human pancreas RNA (25 pg) and total human prostate RNA (25 pg). RT primer was added at 20, 35, or 50 nM. qPCRs were prepared with FW primer at 0.1, 0.35, and 0.6 μM and the universal reverse primer at 0.1, 0.35 and 0.6 μM final. Results are shown in Tables 11 and 12.

The results demonstrate that the ranges of concentrations evaluated here for all three primers can be used to achieve accurate quantification of the two miRNAs with the methods of the invention. For amplification of hsa-miR-205, RT primer concentrations of 35 or 50 nM were generally more effective at achieving higher ΔCts (vs NTC) than a primer concentration of 20 nM. This was less apparent for hsa-miR-16.

TABLE 11
Evaluation of RT primer concentration for amplification of hsa-miR-205 from
pooled human RNA.

| NTC | | | | | Total RNA | | | | | |
|---|---|---|---|---|---|---|---|---|---|---|
| qPCR Primer | | RT | | | qPCR Primer | | RT | | | |
| [μM] | | Primer | | | [μM] | | Primer | | | Δ |
| UR | FW | [nM] | AVG Ct | S.D. | UR | FW | [nM] | AVG Ct | S.D. | Ct |
| 0.1 | 0.1 | 50 | 40.00 | 0.00 | 0.1 | 0.1 | 50 | 35.02 | 0.81 | 4.98 |
| | | 35 | 40.00 | 0.00 | | | 35 | 34.82 | 0.62 | 5.18 |
| | | 20 | 40.00 | 0.00 | | | 20 | 37.25 | 3.90 | 2.75 |
| | 0.35 | 50 | 40.00 | 0.00 | | 0.35 | 50 | 34.08 | 0.67 | 5.92 |
| | | 35 | 40.00 | 0.00 | | | 35 | 34.46 | 0.43 | 5.54 |
| | | 20 | 40.00 | 0.00 | | | 20 | 34.15 | 0.13 | 5.85 |
| | 0.6 | 50 | 40.00 | 0.00 | | 0.6 | 50 | 33.54 | 0.17 | 6.46 |
| | | 35 | 40.00 | 0.00 | | | 35 | 33.52 | 0.30 | 6.48 |
| | | 20 | 40.00 | 0.00 | | | 20 | 35.08 | 1.51 | 4.92 |
| 0.35 | 0.1 | 50 | 40.00 | 0.00 | 0.35 | 0.1 | 50 | 37.21 | 1.25 | 2.79 |
| | | 35 | 40.00 | 0.00 | | | 35 | 36.90 | 1.03 | 3.10 |
| | | 20 | 39.14 | 1.21 | | | 20 | 35.94 | 1.14 | 3.21 |
| | 0.35 | 50 | 40.00 | 0.00 | | 0.35 | 50 | 33.78 | 0.26 | 6.22 |
| | | 35 | 40.00 | 0.00 | | | 35 | 33.76 | 0.33 | 6.24 |
| | | 20 | 40.00 | 0.00 | | | 20 | 35.74 | 1.77 | 4.26 |
| | 0.6 | 50 | 40.00 | 0.00 | | 0.6 | 50 | 33.30 | 0.81 | 6.70 |
| | | 35 | 40.00 | 0.00 | | | 35 | 32.91 | 0.39 | 7.09 |
| | | 20 | 40.00 | 0.00 | | | 20 | 34.46 | 0.85 | 5.54 |
| 0.6 | 0.1 | 50 | 40.00 | 0.00 | 0.6 | 0.1 | 50 | 35.70 | 1.10 | 4.30 |
| | | 35 | 40.00 | 0.00 | | | 35 | 36.46 | 0.76 | 3.54 |
| | | 20 | 40.00 | 0.00 | | | 20 | 39.20 | 0.00 | 0.80 |
| | 0.35 | 50 | 40.00 | 0.00 | | 0.35 | 50 | 34.21 | 0.05 | 5.79 |
| | | 35 | 40.00 | 0.00 | | | 35 | 33.86 | 0.24 | 6.14 |
| | | 20 | 40.00 | 0.00 | | | 20 | 35.38 | 1.58 | 4.62 |
| | 0.6 | 50 | 40.00 | 0.00 | | 0.6 | 50 | 33.13 | 0.36 | 6.87 |
| | | 35 | 40.00 | 0.00 | | | 35 | 34.61 | 0.05 | 5.39 |
| | | 20 | 40.00 | 0.00 | | | 20 | 34.56 | 1.08 | 5.44 |

NTC, no target control.
UR, universal reverse qPCR primer.
FW, forward qPCR primer.
AVG Ct, average Ct of duplicate qPCRs.
S.D., standard deviation.
ΔCt, Ct (NTC) − Ct (total RNA).

TABLE 12
Evaluation of RT primer concentration for amplification
of hsa-miR-16 from pooled human RNA.

| NTC | | | | | Total RNA | | | | | |
|---|---|---|---|---|---|---|---|---|---|---|
| qPCR Primer | | RT | | | qPCR Primer | | RT | | | |
| [μM] | | Primer | | | [μM] | | Primer | | | Δ |
| UR | FW | [nM] | AVG Ct | S.D. | UR | FWD | [nM] | AVG Ct | S.D. | Ct |
| 0.1 | 0.1 | 50 | 40.00 | 0.00 | 0.1 | 0.1 | 50 | 30.37 | 0.22 | 9.63 |
| | | 35 | 40.00 | 0.00 | | | 35 | 30.58 | 0.13 | 9.42 |
| | | 20 | 40.00 | 0.00 | | | 20 | 30.89 | 0.15 | 9.11 |
| | 0.35 | 50 | 40.00 | 0.00 | | 0.35 | 50 | 28.63 | 0.07 | 11.37 |
| | | 35 | 38.53 | 2.08 | | | 35 | 28.41 | 0.04 | 10.12 |
| | | 20 | 40.00 | 0.00 | | | 20 | 28.85 | 0.20 | 11.15 |
| | 0.6 | 50 | 40.00 | 0.00 | | 0.6 | 50 | 28.29 | 0.07 | 11.71 |
| | | 35 | 38.39 | 2.27 | | | 35 | 28.54 | 0.40 | 9.85 |
| | | 20 | 40.00 | 0.00 | | | 20 | 29.40 | 0.42 | 10.60 |
| 0.35 | 0.1 | 50 | 40.00 | 0.00 | 0.35 | 0.1 | 50 | 30.54 | 0.20 | 9.46 |
| | | 35 | 40.00 | 0.00 | | | 35 | 30.75 | 0.27 | 9.25 |
| | | 20 | 39.14 | 1.21 | | | 20 | 31.23 | 0.30 | 8.77 |
| | 0.35 | 50 | 40.00 | 0.00 | | 0.35 | 50 | 28.66 | 0.13 | 11.34 |
| | | 35 | 40.00 | 0.00 | | | 35 | 29.04 | 0.08 | 10.96 |
| | | 20 | 40.00 | 0.00 | | | 20 | 29.14 | 0.01 | 10.86 |
| | 0.6 | 50 | 40.00 | 0.00 | | 0.6 | 50 | 28.20 | 0.01 | 11.80 |
| | | 35 | 40.00 | 0.00 | | | 35 | 28.35 | 0.04 | 11.65 |
| | | 20 | 40.00 | 0.00 | | | 20 | 28.70 | 0.02 | 11.30 |
| 0.6 | 0.1 | 50 | 40.00 | 0.00 | 0.6 | 0.1 | 50 | 30.73 | 0.04 | 9.27 |
| | | 35 | 40.00 | 0.00 | | | 35 | 30.75 | 0.14 | 9.25 |
| | | 20 | 40.00 | 0.00 | | | 20 | 31.52 | 0.24 | 8.48 |
| | 0.35 | 50 | 40.00 | 0.00 | | 0.35 | 50 | 29.17 | 0.02 | 10.83 |
| | | 35 | 40.00 | 0.00 | | | 35 | 29.24 | 0.40 | 10.76 |
| | | 20 | 40.00 | 0.00 | | | 20 | 29.43 | 0.15 | 10.57 |
| | 0.6 | 50 | 40.00 | 0.00 | | 0.6 | 50 | 28.56 | 0.18 | 11.44 |
| | | 35 | 40.00 | 0.00 | | | 35 | 28.62 | 0.20 | 11.38 |
| | | 20 | 40.00 | 0.00 | | | 20 | 29.05 | 0.35 | 10.95 |

NTC, no target control.
UR, universal reverse qPCR primer.
FW, forward qPCR primer.
AVG Ct, average Ct of duplicate qPCRs.
S.D., standard deviation.
ΔCt, Ct (NTC) − Ct (total RNA).

### Example 10

### Evaluation of qPCR Forward Primer Concentration

Various concentrations of qPCR FW primer were evaluated to amplify and quantify six different miRNA targets from total human RNA. For RT, pooled total human RNA (75 pg per reaction) consisted of a pooled mixture of total human pancreas RNA (25 pg), total human prostate RNA (25 pg), and total human lung RNA (25 pg). RT reactions were prepared with MMLV RT (10 U/r×n) and RT primer (50 nM) and incubated at 16° C. for 20 min, then at 42° C. for 20 min, and finally at 95° C. for 10 min in a GeneAmp PCR system 9700 (Applied Biosystems). No target control (NTC) reactions were prepared with nuclease free water in place of total RNA. For qPCRs, 3 μL of the RT reactions were transferred into 15 μL qPCRs. qPCR FW primers were evaluated at three different concentrations (0.3, 0.4, and 0.5 μM). The qPCR universal reverse primer was present at 0.5 μM, the universal TaqMan probe at 80 nM, and PlatinumTaq (Invitrogen) at 0.033 U/μL. qPCRs were performed in a “standard” mode and in a “fast” mode. For standard qPCR, reactions were incubated at 95° C. for 5 min, then at 95° C. for 15 sec and 60° C. for 1 min for 40 cycles in a 7900HT Fast Real-Time PCR System (Applied Biosystems). For fast qPCR, reactions were incubated at 95° C. for 1 min, then at 95° C. for 3 sec and 60° C. for 30 sec for 40 cycles in a 7500 Fast Real-Time PCR System (Applied Biosystems). Results of these experiments are shown in Table 13.

The results demonstrate that the range of concentrations evaluated here for the qPCR FW primer can be used to achieve accurate quantification of the six miRNAs with the methods of the invention. The optimal FW primer concentration was found to be target dependent. In general, for the targets evaluated here, a qPCR FW primer concentration of 0.3 μM was found to yield a ΔCt (vs NTC) that was at or near the maximum for the three concentrations evaluated.

TABLE 13
Evaluation of forward primer concentration for the quantification
of six different miRNAs in pooled human total RNA. FW, qPCR
forward primer. ΔCt, Ct (NTC) - Ct (Total
RNA).

| | |
|---|---|
| Standard PCR | Fast PCR |

| miRNA | FW [μM] | ΔCt | FW [μM] | ΔCt |
|---|---|---|---|---|
| hsa-miR-16 | 0.3 | 9.32 | 0.3 | 10.23 |
| | 0.4 | 9.22 | 0.4 | 9.19 |
| | 0.5 | 9.22 | 0.5 | 9.99 |
| hsa-miR-375 | 0.3 | 15.70 | 0.3 | 12.65 |
| | 0.4 | 11.95 | 0.4 | 10.71 |
| | 0.5 | 12.33 | 0.5 | 8.52 |
| hsa-miR-143 | 0.3 | 12.83 | 0.3 | 17.29 |
| | 0.4 | 18.06 | 0.4 | 17.86 |
| | 0.5 | 18.11 | 0.5 | 17.97 |
| hsa-miR-200c | 0.3 | 17.16 | 0.3 | 12.65 |
| | 0.4 | 15.59 | 0.4 | 10.71 |
| | 0.5 | 16.46 | 0.5 | 8.52 |
| hsa-miR-203 | 0.3 | 5.96 | 0.3 | 8.89 |
| | 0.4 | 6.58 | 0.4 | 3.02 |
| | 0.5 | 4.70 | 0.5 | 2.19 |
| let-7a | 0.3 | 19.25 | 0.3 | 18.05 |
| | 0.4 | 17.00 | 0.4 | 19.07 |
| | 0.5 | 17.96 | 0.5 | 19.46 |

### Example 11

### Comparison of Hot Start and Conventional DNA Polymerase for qPCR

“Hot-start” or conventional DNA polymerases were compared in quantitative PCR of RNA targets. In general, “hot-start” Taq polymerases are modified to inhibit polymerization activity of the enzyme prior to the start of the PCR. “Hot-start” enzyme modifications are generally inactivated during the initial 95° C. denaturation step of PCR. In these studies, Platinum Taq™ (Invitrogen), a hot-start Taq polymerase and SuperTaq™ (Ambion), a conventional, non-hot-start Taq polymerase were used. RTs and qPCRs were performed as described in Example 1 with the following modifications. Human pancreas total RNA (Ambion) (0.2 ng, 2 ng, or 20 ng) was added to RT reactions as the source of target RNA. RTs were incubated at 16° C. for 30 min, then at 42° C. for 30 min, then at 95° C. for 10 min. qPCRs utilized 0.04 U/μl SuperTaq™ or Platinum Taq, FW primer at 0.87 μM, universal reverse primer at 0.47 μM, and universal TaqMan™ probe at 66 nM. qPCRs were incubated at 95° C. for 5 min, then at 95° C. for 15 sec and 60° C. for 1 min for 40 cycles, in a 7500 Real-Time PCR System (Applied Biosystems). Four different miRNAs were amplified from the target RNA sample and quantified using the assay of the invention. Results are shown in Table 14.

The results demonstrate that the use of a hot-start Taq polymerase (Platinum Taq), provides better specificity of detection in the methods of the invention. This is indicated by the higher Ct values for the no target control (NTC) samples when compared with the corresponding values for the conventional Taq polymerase (SuperTaq). The use of the hot-start polymerase also generally results in higher sensitivity of detection as represented by a larger ACt when low amounts of target RNA are present (Avg Ct [NTC]-Avg Ct [0.2 ng]). The uniformity of the slope of the Ct values among samples amplified with Platinum Taq is superior to that of samples amplified with conventional Taq polymerase, indicating the improved robustness and accuracy achieved with the hot-start polymerase.

TABLE 14
Comparison of “hot-start” and conventional Taq polymerases in qPCRs for
quantification of four different miRNAs.

| | RT | | | | | | | | | | | |
|---|---|---|---|---|---|---|---|---|---|---|---|---|
| | Primer/ | | Avg | | Avg | | | Avg | | Avg | | |
| | FWD | Taq | Ct | S.D. | Ct | S.D. | ΔCt | Ct | S.D. | Ct | S.D. | |
| miRNA | Primer | Polymerase | NTC | NTC | 0.2 ng | 0.2 ng | 0.2 ng | 2 ng | 2 ng | 20 ng | 20 ng | Slope |
| Has- | RT 6/ | Platinum | 37.86 | 1.82 | 35.28 | 2.11 | 2.58 | 32.03 | 0.34 | 29.26 | 0.37 | −3.01 |
| miR-143 | FW 17 | Taq | | | | | | | | | | |
| Hsa- | RT 6/ | SuperTaq | 29.72 | 1.48 | 27.05 | 1.20 | 2.67 | 26.10 | 0.15 | 25.78 | 0.17 | −0.43 |
| miR-143 | FW 17 | | | | | | | | | | | |
| Hsa- | RT 7/ | Platinum | 40 | 0.00 | 29.72 | 0.61 | 10.28 | 26.27 | 0.16 | 23.45 | 0.21 | −3.13 |
| miR-194 | FW 17 | Taq | | | | | | | | | | |
| Hsa- | RT 7/ | SuperTaq | 36.76 | 2.29 | 28.81 | 0.06 | 7.95 | 25.60 | 0.01 | 22.74 | 0.15 | −3.03 |
| miR-194 | FW 17 | | | | | | | | | | | |
| Hsa- | RT 8/ | Platinum | 39.03 | 1.36 | 31.17 | 0.18 | 7.86 | 27.63 | 0.20 | 24.56 | 0.16 | −3.31 |
| miR-217 | FW 16 | Taq | | | | | | | | | | |
| Hsa- | RT 8/ | SuperTaq | 34.63 | 3.73 | 30.13 | 0.01 | 4.50 | 26.84 | 0.01 | 23.77 | 0.04 | −3.18 |
| miR-217 | FW 16 | | | | | | | | | | | |
| Hsa- | RT 6/ | Platinum | 40 | 0.00 | 28.78 | 0.06 | 11.22 | 25.10 | 0.09 | 22.26 | 0.01 | −3.26 |
| miR-375 | FW 15 | Taq | | | | | | | | | | |
| Hsa- | RT 6/ | SuperTaq | 37.37 | 0.91 | 27.34 | 0.01 | 10.03 | 24.24 | 0.18 | 21.62 | 0.06 | −2.85 |
| miR-375 | FW 15 | | | | | | | | | | | |

Avg Ct. average of duplicate reactions.
S.D., standard deviation.
ΔCt, Ct (NTC) − Ct (Total RNA).

### Example 12

### Evaluation of Different Hot-Start DNA Polymerases in qPCR

These studies were undertaken to evaluate different types of hot-start polymerases. Two hot-start DNA polymerases were used in these studies. Platinum® Taq DNA polymerase (Invitrogen) is an enzyme mixture composed of recombinant Taq DNA polymerase, Pyrococcus species GB-D polymerase, and Platinum® Taq antibody. Pyrococcus species GB-D polymerase possesses proofreading ability and increases fidelity approximately six-fold when mixed with Taq polymerase. An anti-Taq DNA polymerase antibody provides “hot-start” activity by binding to the Taq polymerase and inhibiting its activity until the temperature of the reaction reaches 94° C. HotStart-IT™ Taq DNA polymerase (USB) uses a different hot-start method that relies on primer sequestration. Here a recombinant protein has been added to Taq polymerase to bind and sequester PCR primers at lower temperatures, making them unavailable for use by the Taq polymerase. Following the initial denaturation step during PCR, the protein is inactivated and the primers are free to participate in the amplification reaction.

Reverse transcriptions and qPCRs were performed as described in Example 1, using either Platinum® Taq or HotStart IT™ in the qPCRs. Reverse transcriptions used pooled human total RNA (75 pg) consisting of a mixture of human pancreas, prostate, and lung total RNAs (25 ng each). In no target control (NTC) reactions, total RNA was replaced with nuclease-free water. Six different miRNAs were amplified from the target RNA sample and quantified using the assay of the invention. Results are shown in Table 15.

TABLE 15
Comparison of two different “hot-start” DNA polymerases
for use in qPCR.

| | Platinum Taq | | | HotStart-IT | | |
|---|---|---|---|---|---|---|
| | Avg Ct | | | Avg Ct | | |
| miRNA | NTC | 75 pg RNA | ΔCt | NTC | 75 pg RNA | ΔCt |
| Has-miR-16 | 36.19 | 27.21 | 8.98 | 35.54 | 26.37 | 9.17 |
| Hsa-miR-143 | 40.00 | 26.58 | 13.42 | 40.00 | 25.13 | 14.87 |
| Hsa-miR-375 | 38.93 | 26.68 | 12.25 | 38.62 | 26.38 | 12.24 |
| hsa-miR-200c | 40.00 | 27.22 | 12.78 | 39.66 | 27.40 | 12.26 |
| Hsa-miR-203 | 40.00 | 33.43 | 6.58 | 36.42 | 31.72 | 4.70 |
| hsa-let-7a | 40.00 | 29.66 | 10.34 | 38.76 | 26.18 | 12.58 |

Avg Ct.. average of duplicate reactions.
S.D., standard deviation.
ΔCt, Ct (NTC) − Ct (Total RNA).

Platinum® Taq and HotStart-IT™ demonstrated similar specificity and sensitivity when used in the methods of the invention to detect and quantify the six miRNAs shown in Table 15. The results demonstrate that various hot-start polymerases are suitable for use in certain embodiments of the invention. In addition, polymerase enzymes that employ different mechanisms for the hot-start process can be used.

### Example 13

### Exonuclease I Treatment of RT Reactions

Escherichia coli exonuclease I catalyzes the removal of nucleotides from single stranded DNA in the 3′ to 5′ direction. It may be used to degrade excess single-stranded primer from a reaction mixture containing double-stranded product, such as a reverse transcription reaction or a PCR. Since the carryover of RT primer from the cDNA synthesis step into the PCR can encourage non-target amplification when there is sufficient complementarity between the RT primer and the FW primer, the inventors reasoned that the enzymatic degradation of the RT primer prior to PCR would enhance specificity for those reactions that demonstrated suboptimal specificity. The following studies were performed to determine if exonuclease I is beneficial for use in the methods of the invention. Reverse transcription reactions and qPCRs were performed as described in Example 1, with the following modifications. Human pancreas total RNA (100 ng) was added to 10 μl RT reactions. Three different miRNAs were amplified from the target RNA sample and quantified using the assay of the invention. RTs used 20 U of SuperScript™ II reverse transcriptase (Invitrogen). Reactions were incubated at 16° C. for 20 min, then at 42° C. for 20 min. Various amounts of exonuclease I were then added to RT mixtures (Table 16) and incubated further at 37° C. for 30 min, then at 95° C. for 15 min. For qPCR, 3 μl of the RT reaction was added to each qPCR (15 μl final volume), and reactions were incubated as described in Example 1. The results are shown in Table 16.

TABLE 16
Effect of Exonuclease I treatment of reverse transcription
reaction on RNA quantification.

| | | | Average Ct | | |
|---|---|---|---|---|---|
| | | RT Primer | | RNA | |
| Exonuclease I | miRNA target | F̅W̅D̅ P̅r̅i̅m̅e̅r̅ | NTC | 100 pg | ΔCt |
| 0 U | hsa-miR-16 | RT 9/FW 13 | 39.37 | 33.44 | 5.93 |
| | hsa-miR-143 | RT 8/FW 13 | 39.35 | 33.63 | 5.73 |
| | | RT 12/FW 16 | 27.12 | 26.85 | 0.27 |
| | hsa-miR-375 | RT 9/FW 14 | 40.34 | 28.02 | 12.32 |
| | | RT 12/FW 16 | 35.23 | 27.45 | 7.77 |
| 10 U | hsa-miR-16 | RT 9/FW 13 | 41.22 | 33.18 | 8.03 |
| | hsa-miR-143 | RT 8/FW 13 | 42.74 | 34.07 | 8.67 |
| | | RT 12/FW 16 | 32.00 | 29.72 | 2.28 |
| | hsa-miR-375 | RT 9/FW 14 | 50.00 | 28.01 | 21.99 |
| | | RT 12/FW 16 | 36.82 | 27.69 | 9.13 |
| 20 U | hsa-miR-16 | RT 9/FW 13 | 45.58 | 34.26 | 11.33 |
| | hsa-miR-143 | RT 8/FW 13 | 46.27 | 34.56 | 11.70 |
| | | RT 12/EW 16 | 34.79 | 30.03 | 4.76 |
| | hsa-miR-375 | RT 9/FW 14 | 43.73 | 28.99 | 14.74 |
| | | RT 12/FW 16 | 43.65 | 28.30 | 15.35 |

Average Ct, average of duplicate qPCRs.
ΔCt, Ct(NTC) − Ct (100 pg RNA).

The data in Table 16 demonstrate that addition of exonuclease I to RT mixtures, following the RT reaction, can increase the specificity of RNA detection. Discrimination between samples with a target RNA and samples without a target RNA is enhanced. In quantification of three miRNAs, with various RT primer/FW primer combinations, the ACt (Ct [NTC]-Ct [100 pg RNA]) increased. Specificity is particularly improved by exonuclease I treatment when the ΔCt is small, such as in the case of hsa-miR-143 with primers RT 12 and FW 16.

### Example 14

### Detection of miRNA in Total RNA

These studies were performed to determine if detection of small RNAs is affected by the total RNA background. Synthetic hsa-miR-205 and hsa-miR-372 were serially diluted in nuclease-free water, and 10,000, 1,000 or 0 (NTC) copies were added to 15 μL RT reactions. E. coli Total RNA 0, 2 ng, or 20 ng (Ambion) was added to reverse transcription reactions with hsa-miR-205, and human whole blood total RNA (0, 2 ng, or 200 ng purified with MirVana PARIS™ kit; Ambion) was added to RT reactions with hsa-miR-372. Other reaction components were as described in Example 1. Reactions were incubated at 16° C. for 15 min, then at 42° C. for 15 min, then at 95° C. for 10 min. One fifth (3 μl) of each RT reaction was transferred into a 15 μL qPCR. qPCRs were performed as described in Example 1. Real-time PCR data were analyzed with the SDS 2.3 program (Applied Biosystems). Results are shown in Table 17.

TABLE 17
Detection of miRNAs in a large background amount of total RNA.

| | Copies of synthetic miRNA | | | | | | | | |
|---|---|---|---|---|---|---|---|---|---|
| | 0 (NTC) | | | 1,000 | | | 10,000 | | |
| Total RNA Background | 0 ng | 2 ng | 20 ng | 0 ng | 2 ng | 20 ng | 0 ng | 2 ng | 20 ng |
| hsa-miR-205 (Avg Ct) | 38.35 | 38.33 | 40.00 | 32.40 | 32.49 | 32.48 | 29.13 | 29.24 | 29.18 |
| hsa-miR-372 (Avg Ct) | 38.48 | 39.24 | 37.95 | 32.87 | 33.09 | 32.80 | 28.57 | 28.46 | 29.28 |

The results demonstrate the specific detection of as few as 1,000 copies of each miRNA in a background of as much as 20 ng exogenous total RNA.

### Example 15

### Detection of endogenous miRNAS in Human Total RNA

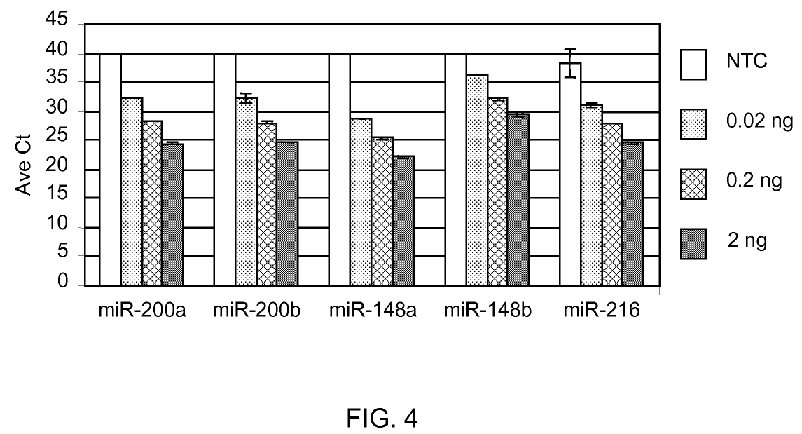

The methods of the invention are capable of detection and quantification of endogenous miRNAs in a total RNA sample. In this example, human pancreas total RNA (0 ng (NTC), 0.02 ng, 0.2 ng or 2 ng) served as a source of target miRNAs in 15 μL RT reactions. RT reactions were prepared with MMLV RT (10 U/r×n). Other reagents were at the concentrations described in Example 1. RTs were incubated at 16° C. for 30 min, then at 42° C. for 30 min, then at 95° C. for 10 min. One sixth of the reaction (2.5 μL) was transferred into a 15 μL qPCR. For qPCR, the FW primer final concentration was 0.87 μM, the universal reverse primer final concentration was 0.47 μM, and the universal TaqMan™ probe final concentration was 67 nM. Platinum® Taq (Invitrogen) was present at 0.04 U/μL. qPCRs were incubated at 95° C. for 5 min, then at 95° C. for 15 sec and 60° C. for 1 min for 40 cycles in a 7900HT Fast Real-Time PCR System (Applied Biosystems). Real-time PCR data were analyzed with the SDS 2.3 program (Applied Biosystems). The results are shown in Table 18 and FIG. 4.

The results of this study demonstrate that the methods of the invention are capable of specifically detecting endogenous miRNAs in a sample of total RNA. Five different miRNAs were quantified using as little as 20 pg of input total RNA. As provided in the table 18, the theoretical limit of detection (LOD) is the amount of RNA wherein the target can theoretically be detected if one extrapolates from the standard curve and allows 3.3 Ct separation from the NTC (such that the signal would be at least 90% dominated by the presence of the specific target). It is a mathematical construct of convenience that allows different assays run with different amounts of total RNA to be compared more or less directly.

TABLE 18
Detection and quantification of endogenous miRNAs in
human pancreas total RNA.
Data represent average Ct values for duplicate samples.

| | Total RNA Input | | | | |
|---|---|---|---|---|---|
| | 0 | | | | Theoretical |
| Target miRNA | (NTC) | 0.02 ng | 0.2 ng | 2.0 ng | LOD |
| hsa-miR-200a | 40.00 | 32.34 | 28.24 | 24.42 | 1 pg |
| hsa-miR-200b | 40.00 | 32.38 | 28.01 | 24.58 | 1 pg |
| hsa-miR-148a | 40.00 | 28.69 | 25.32 | 22.13 | 80 fg |
| hsa-miR-148b | 40.00 | 36.41 | 32.06 | 29.41 | 17 pg |
| hsa-miR-216 | 38.28 | 31.14 | 27.89 | 24.60 | 2 pg |

### Example 16

### Quantification of Mature miRNA

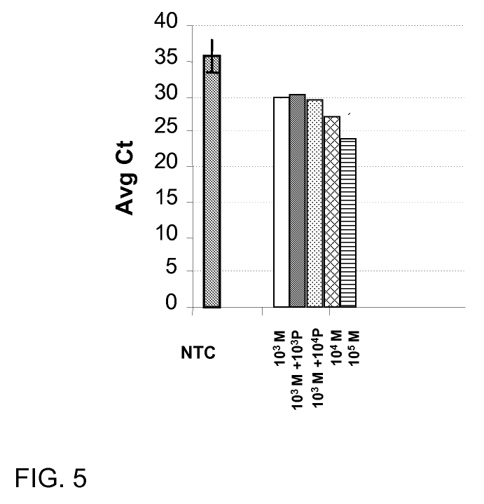

These studies were performed to determine if the precursor form of a miRNA has an effect on the quantification of the mature form of a miRNA. Reverse transcription reactions were assembled as described in Example 1. Synthetic mature hsa-miR-375 and the stem-loop precursor of hsa-miR-375 (Ambion) were serially diluted in nuclease-free water and added to 15 μL RT reactions. Primer RT 9 (50 nM final) was used in RTs. RTs were incubated at 16° C. for 30 min, then at 42° C. for 30 min, then at 95° C. for 10 min. One sixth of the RT reactions (2.5 μL) was transferred into 15 μL qPCRs. qPCRs used primer FW 14 at 0.87 μM, universal reverse primer at 0.47 μM, universal TaqMan probe at 67 nM, and Platinum® Taq (Invitrogen) at 0.04 U/μL. qPCRs were incubated at 95° C. for 5 min, then at 95° C. for 15 sec and 60° C. for 1 min for 40 cycles in a 7900HT Fast Real-Time PCR System (Applied Biosystems). Real-time PCR data were analyzed with the SDS 2.3 program (Applied Biosystems). Results are shown in FIG. 5.

### Example 17

### Evaluation of RNA Assays in the Presence of Genomic DNA

Amplification of no template control samples was compared to amplification of samples containing only human genomic DNA (Promega, G304A) at 30 ng/10 μL RT r×n. RT and qPCR protocols were as described in Example 1. RT primers and FW primers designed to anneal with hsa-miR-16, -21, -26b, -143, and -375 were used for these studies. Results are shown in Table 19.

The results demonstrate that Ct values for the NTC samples are all above 40.00. The results also demonstrate that the Ct values for genomic DNA samples are all near or above 37.00 and represent acceptable Ct values for negative control samples.

TABLE 19
Effects of genomic DNA on RNA quantification.

Avg Ct

| miRNA Specific Primers | NTC | 30 ng genomic DNA |
|---|---|---|
| hsa-miR-16 | 46.39 | 38.34 |
| hsa-miR-21 | 46.21 | 39.49 |
| hsa-miR-26b | 49.31 | 43.99 |
| hsa-miR-143 | 41.30 | 36.88 |
| hsa-miR-375 | 41.16 | 40.94 |

### Example 18

### Quantification of Low Numbers of Small RNAs

Eight different miRNAs were detected and quantified by the methods of the invention. Synthetic hsa-miRs (10², 10³, 10⁴, or 10⁶ copies) were added to RT reactions. RTs and qPCRs were performed as described in Example 1, using 15 μl RT reactions and 15 μl long qPCRs. 7900HT Fast Real-Time PCR System (Applied Biosystems). Real-time PCR data were analyzed with the SDS 2.3 program (Applied Biosystems). Results are shown in Table 20.

TABLE 20
Quantification of 10²–10⁶ copies of eight miRNAs.

| | RT | | | | | | | | | | | |
|---|---|---|---|---|---|---|---|---|---|---|---|---|
| | primer | NTC | | 10² | | 10³ | | 10⁴ | | 10⁶ | | |
| miRNA | FW | Avg | | Avq | | Avg | | Avg | | Avg | | |
| Target | primer | Ct | SD | Ct | SD | Ct | SD | Ct | SD | Ct | SD | Slope |
| hsa-miR-16 | RT 9 | 45.67 | 7.50 | 33.37 | 0.13 | 29.99 | 0.30 | 26.42 | 0.17 | 19.79 | 0.47 | −3.40 |
| | FW 13 | | | | | | | | | | | |
| hsa-miR-21 | RT 10 | 46.50 | 6.06 | 37.79 | 0.28 | 34.78 | 0.57 | 31.18 | 0.21 | 24.30 | 0.12 | −3.40 |
| | FW 13 | | | | | | | | | | | |
| hsa-miR-24 | RT 9 | 38.88 | 0.99 | 30.80 | 0.39 | 27.17 | 0.17 | 23.94 | 0.14 | 17.44 | 0.06 | −3.32 |
| | FW 15 | | | | | | | | | | | |
| hsa-miR-143 | RT 8 | 44.31 | 6.38 | 34.01 | 0.97 | 30.56 | 0.10 | 27.33 | 0.02 | 20.46 | 0.16 | −3.38 |
| | FW 13 | | | | | | | | | | | |
| hsa-miR-148a | RT 10 | 40.90 | 1.92 | 36.45 | 1.12 | 32.98 | 0.13 | 29.94 | 0.39 | 22.88 | 0.10 | −3.38 |
| | FW 12 | | | | | | | | | | | |
| hsa-miR-200c | RT 10 | 45.87 | 7.15 | 33.76 | 0.48 | 31.28 | 0.18 | 27.55 | 0.16 | 20.92 | 0.05 | −3.27 |
| | FW 14 | | | | | | | | | | | |
| hsa-miR-205 | RT 8 | 36.15 | 0.51 | 34.42 | 1.66 | 30.37 | 0.20 | 27.19 | 0.27 | 20.52 | 0.25 | −3.44 |
| | FW 15 | | | | | | | | | | | |
| hsa-miR-375 | RT 9 | 36.42 | 0.45 | 33.02 | 0.58 | 29.88 | 0.06 | 26.60 | 0.10 | 19.68 | 0.08 | −3.34 |
| | FW 14 | | | | | | | | | | | |

The results demonstrate that the materials and methods of certain aspects of the invention are capable of specific and sensitive detection of as low as 100 copies of a miRNA into the reverse transcription reaction. The assays provide specific discrimination between no target control samples and samples with 100 copies of a miRNA. The uniformity of the slopes of the Ct values among the different miRNAs indicate that the methods of the invention are robust and reproducibly accurate and will be broadly applicable to the detection and quantification of small RNAs.

### Example 19

### Effect of Rt Primer Comprising a Universal Reverse Primer

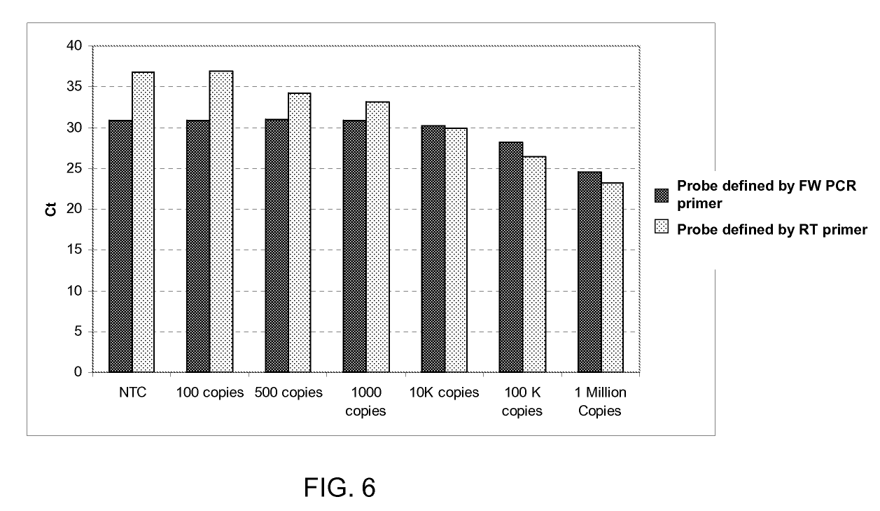

This example demonstrates an improved performance of one aspect of the assay as compared to other assays in which a universal probe is defined by a forward amplification primer. RT primers and FW primers to hsa-miR-375 were designed either with (1) the universal probe sequence defined completely within the RT primer (UPRT) or (2) the universal probe sequence defined within the forward PCR primer (UPFW). Synthetic hsa-miR-375 was then added to the respective RT reactions at inputs ranging from 100 to 1 million copies. For assays that employ aspects of the present invention, optimized conditions for the quantification of hsa-miR-375 were used with the reverse transcription primer, RT 8 and the PCR FW primer, FW 13. When the universal probe was present in the PCR forward primer, an additional universal forward primer was added (as recommended by Rickert et al., 2004). Twenty percent of the RT reaction volume was transferred into the qPCR. As shown in FIG. 6, the assay design that defines the probe within the RT primer sequence was both more sensitive and more specific that the alternative design with respect to the non-template control (NTC) background signal. For example, the non-template control signal was 5.8 Ct's higher for the method of the invention. The consequence of this significantly lower background is that <1000 copies of target could be readily distinguished from the NTC, whereas in the alternative method (wherein the probe is defined by a forward primer), approximately 100-fold more input copies were necessary for the signal to be clearly differentiated from the background. Moreover, once the target-specific signals were acceptably separated from background, the method of the invention was still approximately 3-fold more sensitive. As result, the strategy of embedding the probe sequence in the RT primer enables superior assay performance compared to the alternative approach of defining the probe sequence within a forward primer.

RT Reaction—(1) Assemble components of RT reaction on ice without adding RNA template. This includes 4 U of RNase inhibitor (Ambion) and 10 U of MMLV RT (Ambion) per 10 μl reaction and RT primer (comprising a region of ˜8-11 bases complementarity to the 3′ end of the target miRNA, and a non-complementary region containing a universal dual-labeled fluorescent probe sequence). The final concentration of the RT primer containing the universal probe segment was 50 nM, and separately, the concentration of the RT lacking the universal probe segment was also 50 nM. (2) Add RNA template (1 pg to 40 ng range). If adding a synthetic RNA, add a background of 10 ng/ul Poly A RNA. (3) Incubate RT reaction at 16° C. for 15 min, then 42° C. for 30 minutes, then 10 minutes at 85° C.

PCR Reaction—(1) Assemble RT-PCR reaction components. This can include Platinum® Taq buffer and Platinum® Taq at 0.33 U/μl, and Mg²⁺ at 5 mM. The concentration of the FW primer was 300 nM for those reactions where RT primer defined the universal prove sequence. For those reactions where the forward primer defined the prove, the UPFW was added at 16 nM, whereas the inversal forward primer was added at 300 nM. For all reactions, the Universal reverse primer was 500 nM, and the TazMan prove concentration was 80 nM final. (2) Carry over 1/5 volume of the RT reaction into the PCR reaction. (3) PCR cycles: 95° C. 1 min; then 50 cycles at 95° C. 15 sec and 60° C. 45 sec.

### Example 20

### Sensitivity of Detection in FFPE RNA Increases as the Size of the RNA Amplicon Decreases

FFPE RNA is known to be chemically modified by the fixation process and highly degraded by conventional embedding procedures. As a result, FFPE RNA can evade sensitive quantification by traditional quantification methods, such as microarray analysis or real-time RT-PCR. In both methods, at least 60-600 nucleotide sequences of RNA are queried. It was hypothesized that interrogating even small stretches of RNA—as short as 22 nucleotides—would offer improved detection sensitivity, particularly for highly fragmented FFPE RNA. To study this application, total RNA was isolated using the RecoverAll™ kit (Ambion) from two different FFPE blocks of human myometrium tissue. One block was 7 years old; the other was 11 years old. In both cases, the RNA was highly degraded, as visualized on an RNAchip® using the bioanalyzer 2100 (Agilent).

A series of primers complementary to the human cyclophilin transcript were designed that created amplicon sizes of 180 to 22 nucleotides following RT-PCR using the method of the invention. Each amplicon share a common RT primer; only the target-specific forward primer sequence was varied to change the length of each amplicon. A total of 2 ng of total RNA from each of the two FFPE RNA samples was added to each RT-PCR reaction. Reactions were performed as described in Example 1. As shown in Table 21, the sensitivity of detection of cyclophilin RNA varied by as much as 1000-fold, and, further, this sensitivity increased markedly as the amplicon size was progressively shortened. The detection signal was strongest for the shortest amplicons, namely those that were enabled by the method of the invention. These data demonstrate that amplicons that are significantly shorter than those used in conventional real-time qRT-PCR can improve the detection sensitivity in FFPE RNA by an order of magnitude or greater.

TABLE 21
The sensitivity of the detection of cyclophilin RNA in FFPE
archived tissue increases as the amplicon size decreases.

| | FFPE (7 yr) | FFPE (11 yr) |
|---|---|---|
| 22 nt | 100 | 100 |
| 38 nt | 66 | 63 |
| 63 nt | 22 | 11 |
| 106 nt | 5 | 3 |
| 180 nt | 0.8 | 0.1 |

### REFERENCES

The following references, to the extent that they provide exemplary procedural or other details supplementary to those set forth herein, are specifically incorporated herein by reference.
U.S. Pat. No. 5,188,934
U.S. Pat. No. 5,234,809
U.S. Pat. No. 5,366,860
U.S. Pat. No. 5,432,272
U.S. Pat. No. 5,538,848
U.S. Pat. No. 5,614,365
U.S. Pat. No. 5,789,224
U.S. Pat. No. 5,800,996
U.S. Pat. No. 5,847,162
U.S. Pat. No. 5,863,727
U.S. Pat. No. 5,925,517
U.S. Pat. No. 5,936,087
U.S. Pat. No. 5,945,526
U.S. Pat. No. 5,965,364
U.S. Pat. No. 6,001,983
U.S. Pat. No. 6,008,379
U.S. Pat. No. 6,020,481
U.S. Pat. No. 6,051,719
U.S. Pat. No. 6,103,476
U.S. Pat. No. 6,140,054
U.S. Pat. No. 6,140,500
U.S. Pat. No. 6,150,097
U.S. Pat. No. 6,191,278
U.S. Pat. No. 6,355,421
U.S. Pat. No. 6,383,752
U.S. Pat. No. 6,485,901
U.S. Pat. No. 6,548,250
U.S. Pat. No. 6,589,743
U.S. Pat. No. 6,590,091
U.S. Pat. No. 6,593,091
U.S. Pat. No. 6,596,490
U.S. Pat. No. 7,001,724
U.S. Publn. 20030198944
U.S. Publn. 20030194726
U.S. Publn. 20050266418
Andrus, In: Nucleic Acids in Chemistry and Biology, 2^{nd} Ed., Oxford University Press, 15-81, 1996.
Ausubel et al., In: Current Protocols in Molecular Biology, Volume 1(2), John Wiley & Sons, NY, 1993.
Ausubel et al., In: Current Protocols in Molecular Biology, Greene Publishing and Wiley-Interscience, NY, 1987.
Barringer, et al., Gene, 89:117, 1990.
Brosius et al., Virus Genes, 11: 163-179, 1995.
Broude et al., Trends Biotechnol., 20:249-56, 2002.
Chapter 3 of Laboratory Techniques in Biochemistry and Molecular Biology: Hybridization With Nucleic Acid Probes, Part I. Theory and Nucleic Acid Preparation, Tijssen (Ed.), NY, 1993.
Chemical methods for 5′ non-isotopic labeling of PCR probes and primers, PCR 2: A Practical Approach, Oxford University Press, Oxford, 39-54, 1995.
Denli et al., Trends Biochem. Sci., 28:196, 2003.
Garman, In: Non-Radioactive Labeling, A Practical Introduction, Academic Press, San Diego, 1997.
Girard et al., Nature, 442(7099):199-202, 2006.
Guatelli, et al., Proc. Nat. Acad. Sci. USA, 87: 1874,1990.
Huang et al., Chem. Res. Toxicol., 15:118-126, 2002.
Innis, et al., In: PCR Protocols. A guide to Methods and Application, Academic Press, Inc. San Diego, 1990.
Isacsson et al., Mol. Cell. Probes., 14:321-328, 2000.
Kricka, In: Nonisotopic DNA Probe Techniques, Academic Press, San Diego, 3-28, 1992.
Kubista et al., SPIE, 4264:53-58, 2001.
Kwoh et al., Proc. Natl. Acad. Sci. USA, 86:1173, 1989.
Lagos-Quintana et al., Science, 294(5543):853-858, 2001.
Landegren, et al., Science 241:1077-1080, 1988.
Lau et al., Science, 294(5543):858-862, 2001.
Lee and Ambros, Science, 294(5543):862-864, 2001.
Levin, Cell, 88:5-8, 1997.
Maxwell et al., J. Am. Chem. Soc., 124:9606-9612, 2002.
Mhlanga et al., Methods, 25:463-471, 2001.
Miller et al., Nucleic Acids Res., 16(3):9-10, 1988.
Olsen et al., Dev. Biol., 216:671, 1999.
PCT Appln. WO 00/56748
PCT Appln. WO 00/66604
PCT Appln. WO 97/45539
PCT Appln. WO 99/21881
Riccelli et al., Nucleic Acids Res., 30:4088-4093, 2002.
Rickert et al., Clin. Chem., 50(9):1680-1683, 2004
Saiki et al., Nature, 324:163-166, 1986.
Sambrook et al., In: Molecular cloning: a laboratory manual, 2^{nd} Ed., Cold Spring Harbor Laboratory Press, Cold Spring Harbor, N.Y., 1989.
Schmitt et al., Nucleic Acid Res., 18:3091-3092, 1990.
Seggerson et al., Dev. Biol., 243:215, 2002.
Solinas et al., Nucleic Acids Res., 29:E96, 2001.
Svanvik et al., Anal Biochem., 281:26-35, 2000.
Tsourkas et al., Nucleic Acids Res., 30:4208-4215, 2002.
Tyagi and Kramer, Nat. Biotechnol., 14:303-308, 1996.
Walsh et al, Biotechniques, 10(4):506-513, 1991.
Whitcombe et al., Curr. Opin. Biotechnol., 9(6):602-608, 1998.
Whitcombe et al., Nat. Biotechnol., 17:804-807, 1999.
Wolffs et al., Biotechniques, 766:769-771, 2001.
Wu and Wallace, Genomics, 4:560-569, 1989.
Yu et al, J. Am. Chem. Soc., 14:11155-11161, 2001.
Zhang et al., Shanghai., 34:329-332, 2002.

1. A method of detecting one or more RNA comprising the steps of:
(a) reverse transcribing one or more RNA target using one or more linear reverse transcription primer comprising in a 5′ to 3′ direction
(i) a primer segment,
(ii) a non-target probe segment, and
(iii) a 3′ target specific segment that anneals to a RNA target;
(b) amplifying one or more RNA or RNA segment from all or part of the reverse transcription reaction using a first amplification primer that anneals to the 3′ end of a reverse transcribed RNA target and a second primer that anneals to a sequence complementary to the primer segment; and
(c) detecting amplification of a target nucleic acid.

2. The method of claim 1, wherein the RNA target is present in less than 1,000,000 copies.

3. The method of claim 2, wherein the RNA target is present in less than 1,000 copies.

4. The method of claim 1, wherein the 3′ target specific segment of the reverse transcription primer anneals to a contiguous sequence of 7 or more nucleotides of the RNA target.

5. The method of claim 1, wherein the first amplification primer is present at a concentration of 100 to 1000 nM and the second amplification primer is present at a concentration of 100 to 1000 nM.

6. The method of claim 1, wherein detecting amplification comprises detecting association of a probe with a sequence of the probe segment or a complement to the probe segment.

7. The method of claim 6, wherein the probe is a 5′-exonuclease assay probe, stem-loop molecular beacon, stemless or linear beacon, PNA Molecular Beacon, linear PNA beacon, non-FRET probe, stem-loop and duplex scorpion probe, bulge loop probe, pseudo knot probe, cyclicon, minor groove binding (MGB) probe, hairpin probe, peptide nucleic acid (PNA) light-up probe, self-assembled nanoparticle probe, or ferrocene-modified probe.

8. The method of claim 7, wherein the probe is a 5′exonuclease probe or a beacon probe.

9. The method of claim 1, wherein the RNA is greater than 500 base pairs in length.

10. The method of claim 1, wherein the RNA is 1 kilobase or less in length.

11. The method of claim 10, wherein the RNA is 100 bases or less in length.

12. The method of claim 11, wherein the RNA is 25 bases or less in length.

13. The method of claim 1, wherein the first amplification primer comprises a 5′ non-complementary sequence of 2 to 10 nucleotides.

14. The method of claim 13, wherein the first amplification primer comprises a 5′ non-complimentary sequence of 4 to 8 nucleotides.

15. The method of claim 14, wherein the non-complementary sequence comprises cytosine and guanine residues.

16. The method of claim 1, further comprising diluting the reverse transcription reaction prior to amplification.

17. The method of claim 16, wherein the reverse transcription reaction is diluted 2 to 100 fold.

18. The method of claim 1, wherein the RNA is from a biopsy sample, a histological sample, or a biological fluid.

19. The method of claim 18, wherein the histological sample is a formalin or formaldehyde fixed paraffin embedded (FFPE) sample.

20. A method of detecting an miRNA in a sample comprising the steps of:
(a) obtaining a RNA sample;
(b) reverse transcribing one or more miRNA target in the RNA sample using one or more linear reverse transcription primer comprising in a 5′ to 3′ direction
(i) a primer segment,
(ii) a non-target probe segment, and
(iii) a 3′ target specific segment that anneals to a miRNA target;
(c) amplifying the product of the reverse transcription reaction using a first primer that anneals to the 3′ portion of a reverse transcribed target miRNA and a second primer that anneals to a sequence complementary to the primer segment; and
(d) detecting amplification of the probe segment.

21. The method of claim 20, wherein the sample is a biopsy sample, a histological sample, or a biological fluid.

22. The method of claim 21, wherein the histological sample is a FFPE sample.

23. A method of assessing a pathological condition comprising the steps of:
(a) reverse transcribing RNA in a RNA sample from a subject having, suspected of having, or at risk of developing a pathological condition using a reverse transcription primer specific for one or more RNA associated with one or more pathological condition using one or more linear reverse transcription primer comprising in a 5′ to 3′ direction
(i) a universal primer segment,
(ii) a non-target probe segment, and
(iii) a 3′ target specific segment that anneals to a RNA target;
(b) amplifying the product of the reverse transcription reaction using a first primer that anneals to the 5′ portion of a target RNA and a second primer that anneals to the universal primer segment of the reverse transcription primer; and
(c) detecting amplification of the probe segment of the reverse transcription primer.

24. The method of claim 23, wherein the RNA is a miRNA.

25. The method of claim 23, wherein the sample is a biopsy sample, a histological sample, or a biological fluid.

26. The method of claim 25, wherein the histological sample is a FFPE sample.

27-29. (canceled)
